(19) United States
(12) Patent Application Publication
(10) Pub. No.: US 2015/0050368 A1
(43) Pub. Date: Feb. 19, 2015

(54) METHOD FOR PRODUCING A PLANT EXTRACT FROM DESMODIUM AND ITS EXTRACT THEREOF

(71) Applicant: FRANCIS MAES N.V., Oostende (BE)

(72) Inventors: Francis Maes, Oostende (BE); Luc Pieters, Antwerpen (BE); Arnold Vlietinck, Kalmthout (BE); Sandra Apers, Puurs (BE); Nina Hermans, Zoersel (BE)

(21) Appl. No.: 14/386,237

(22) PCT Filed: Mar. 20, 2013
(86) PCT No.: PCT/BE2013/000014

§ 371 Date: Sep. 18, 2014

(30) Foreign Application Priority Data
Mar. 20, 2012 (DE) 2012/0195

(51) Int. Cl. A61K 36/48; A23L 1/30; A23L 2/52; A61K 31/075

(52) U.S. Cl. CPC A61K 36/48; A61K 31/075; A23L 1/3002; A23L 2/52; A23V 2002/00
USPC 424/757; 568/670

(57) ABSTRACT

This invention relates to a method for producing a plant extract, quantified on pinitol, wherein a plant Desmodium adscendens is selected from the Desmodium family, wherein a fraction is extracted from Desmodium plant parts, wherein a plant extract is derived from said fraction thereof, which is remarkable in that a characterised extract is derived from Desmodium adscendens, from which a preparation of said plant extract is quantified on pinitol.

### FIELD OF THE INVENTION

The present invention relates to a method for producing a plant extract from Desmodium, and more specifically Desmodium adscendens.

### BACKGROUND OF THE INVENTION

Since ancient times, plants are used for their medicinal properties. Many present drugs are active components of plants such as paclitaxel (Taxol) of Taxus brevifolia and morphine of Papaver somniferum, or are derived therefrom. In traditional medicine in Africa as well, many plants are used, including Desmodium adscendens. Indeed, in terms of geographical distribution and use, the plant is native to many tropical and subtropical countries of Africa and also South America among others, and it grows on open plains, meadows and along the way, making this plant freely accessible. This makes this plant attractive as a drug for populations where medication is often difficult to purchase due to their cost. So, the plant is used in traditional medicine, i.a. for asthma, pain, fever, epilepsy, hepatitis and muscle spasms. For this, a decoct is used as a hot aqueous extract of the leaves, branches or stems.

There are some commercial preparations of flavonoid containing leaf extracts of this plant in circulation that are marketed as dietary supplements with several properties that promote a good health. Also for the treatment of liver diseases, medicinal plants are being used for centuries. So Silybum marianum, Picrorrhiza kurroa, Curcuma longa, Glycyrrhiza glabra, Phyllantus amarus and Andrographis particulate are plants which were suspected to have a hepatoprotective effect. The liver is a very important organ in the human body that ensures many important tasks, making liver disorders have a major impact on the body. Indeed, cirrhosis of the liver is one of the major causes of death in the Western world. Further, jaundice is a disorder which occurs because of an increase in non-conjugated or indirect bilirubin, or conjugated or direct bilirubin, and causes yellow discolouration of the skin and sclerae. Cirrhosis is a result of progressive necrosis with scarring (fibrosis), with nodular regeneration occurring between the scars and damaging the normal structure. This results in congestion, portal hypertension, encephalopathy, ascites, etc. From 60 to 70% of liver cirrhosis cases are caused by alcohol abuse. Other causes are viral hepatitis, biliary disease and primary haemochromatosis. Liver failure is the end stage of massive necrosis of the liver caused by viral hepatitis, drugs and chemicals, chronic liver diseases, and of hepatic dysfunction without necrosis, such as Reye syndrome.

To understand the mechanisms of liver injury and to gain insight into the degree of cellular injury, blood parameters are measured, for example the transaminases alanine aminotransferase (ALT) and aspartate aminotransferase (AST). ALT is only present in the cytoplasm, while AST also occurs in the mitochondria. The transaminases rise rapidly, since they are released even during cell wall injury. AST rises later than ALT and indicates that more serious necrosis is occurring. Membrane enzymes such as gamma-glutamyl transferase (γGT) and alkaline phosphatase (AP) are present in the cell membranes of almost all of the body's cells. Gamma-GT is elevated in the case of viral or bacterial hepatitis or in the case of intoxication by drugs or alcohol. Alkaline phosphatase is also determined for the diagnosis and follow-up of liver disorders. These parameters provide a picture of the liver injury.

The plant in question Desmodium adscendens notably includes flavonoids such as vitexin (1), rutin (2) and isovitexin, the tetrahydroisochinoline salsoline (3), soyasaponins including soyasaponin I (4), β-phenylethylamines such as tyramine (5) and hordenine (6) shown below under (1) to (6) resp. and an indol-3-alkylamine.

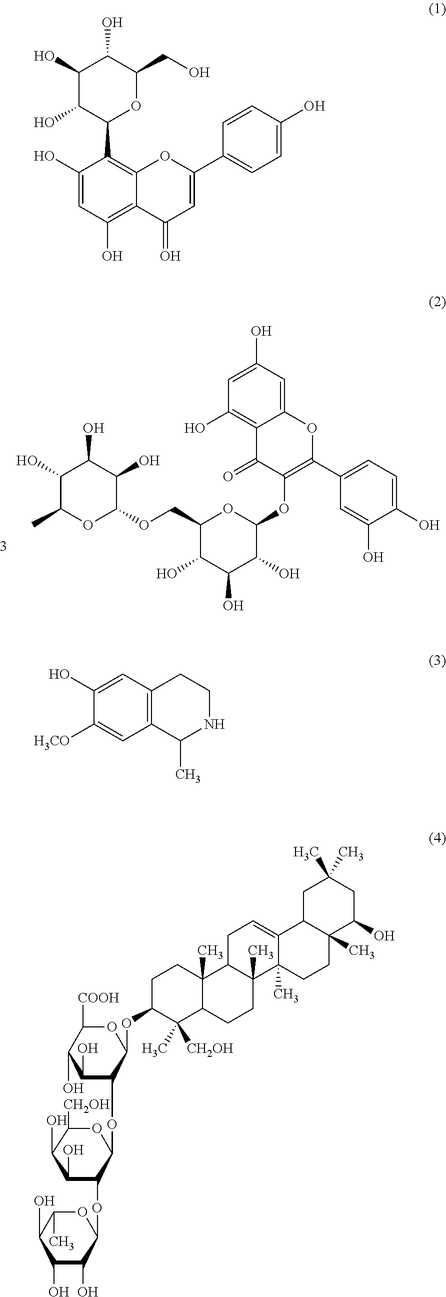

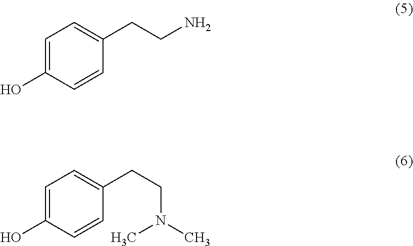

### PRIOR ART

The scientific article MUANDA et at 2011 deals with Desmodium adscendens leaves. It indicates a positive effect in case of infections of the liver. An analytical method is yet indicted in which phenolic compounds are identified, and it mentions the presence of certain ingredients but others not, however, despite the central location they will have in this development as will appear further, which therefore puts the importance of this document strongly at issue here, at least based on the crucial constituent referred to here.

Document EP 0309342 A1 of TUBERY describes the use of Desmodium in the treatment of viral hepatitis of type A or B and toxic hepatitis, but a priori not against all other liver diseases, and drugs for this, in particular for addicted people or cancer treatment. So this document yet also relates to Desmodium, particularly Desmodium adscendens, with the liver as a target organ for use. A decoct is also mentioned as a form of use, in addition to powder from the dried plant. This document further mentions the principal element that Desmodium contains indole alkaloid as an active ingredient. More specifically, this document only relates to an uncharacterized extract of D. adscendens. It provides no further qualitative or quantitative information about the indolalkaloids. In other words, this document provides no validated analytical method for the quantification of the intended crucial constituent here matter. In addition, this document presents D. adscendens only as a drug for the treatment of hepatitis.

Until now, liver diseases were very difficult to treat on the one hand, and even almost impossible to avoid, on the other hand. So this is also the problem raised here: how to prevent these liver diseases.

### AIM OF THE INVENTION

A principal object of this invention is therefore in the first instance the preparation of an quantified extract from Desmodium adscendens that can bring a solutions to the abovementioned problem In this respect, the hepatoprotective properties of extracts of Desmodium adscendens are investigated. When looking for a suitable substance for liver diseases, a substance from the group of inositols was tested. These represent a class of compounds which contain hexahydroxycyclohexane derivatives, in particular cyclic sugar alcohols.

Inositols form a part of the ordinary human diet like sugars, without being toxic. Several studies, including in humans, have shown that D-pinitol exerts an effect which is analogous to that of insulin in order to improve the required control of the glycaemy. Said studies indeed suggest that there appears to be a synergy between D-pinitol and insulin at submaximal concentrations, which is not obvious however in glucose transport.

D-pinitol is a cyclic sugar alcohol having a low molecular weight. It is a methyl derivative of D-chiro-inositol. Both D-pinitol and D-chiroinositol are structurally related to phosphatidyl inositols, which form a link in the insulin-induced signal transduction. D-pinitol, whose structure is depicted below, occurs in many vegetables, soy products and pine trees—but not exclusively- and it is metabolised in the body to chiroinositol.

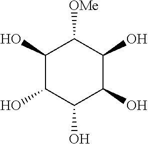 [SMILES: CO[C@H]1[C@H](O)[C@@H](O)[C@@H](O)[C@H](O)[C@@H]1O]

D-pinitol differs from the other inositols because of its specific activity. It is to be understood here that both isomers are meant, thus including the inactive L-pinitol.

A further difference of D-pinitol in regard of the other inositols consists in that it contains a methyl ether group. Given said composition, D-pinitol has a natural influence on the glucose metabolism, and hence on diabetes. In vitro pharmacological properties suggest indeed efficacy of D-pinitol including hypoglycemic, antiatherogenic activity and an influence on the immune system. As far as the hypoglycemic activity is concerned, D-pinitol can improve glucose transport and insulin sensitivity. This is owing to the fact that D-pinitol in humans is metabolised partly to D-chiro-inositol, which is actually the material that is responsible for the biological activity: D-chiroinositol has an effect on diabetes which is formed by partial metabolism of D-pinitol, the active compound for the positive effects, notably in type-2 diabetes. In other words, insulin sensitivity or resistance is improved. Indeed, it is shown that D-pinitol in man is metabolised to D-chiro-inositol, esp in type-2 diabetes, and it is also extracted unchanged.

In other words, it cannot be deduced at first glance that D-pinitol should come under consideration a priori as useful, or even less as an important element for providing a significant contribution to the prevention of the liver diseases discussed here or its treatment.

### SUMMARY OF THE INVENTION

Thus according to the invention, a method is proposed for producing a plant extract, quantified for pinitol, wherein a plant from the Desmodium family is selected, a fraction is extracted from the Desmodium plant parts, and a plant extract is derived from said fraction, which is remarkable in that Desmodium adscendens is selected, from which a characterised extract is derived, from which a preparation of this plant extract is quantified for pinitol.

According to this invention, pinitol is proposed as an active main constituent, which plays a crucial role and thus occupies a central position, especially D-pinitol.

MUANDA however mentions no way D-pinitol as constituent of D. adscendens.

In contrast, however, document WO 2004/084875 A1 of AMICOGEN does relate to pinitol, or a plant extract containing pinitol for the protection of the liver. However, this document mentions plants as soybean, pine, Hovenia dulcis, Acanthopanax senticosus and carob, but again neither Desmodium, nor even less D. adscendens. Furthermore, this document deals only about uncharacterized extracts. There is no qualitative or quantitative information provided on composition, but only that they contain pinitol or chiroinositol.

The article of BEVERIDGE et al, Aust. J. Chem., 1977, 30, 1583-1590 yet reports the analysis by gas chromatography of D-pinitol in two Desmodium types. But this article is about D. intortum and D. uncinatum, two species that are botanically different from D. adscendens.

In an analogous way, the article of FORD et al, Aust. J. Agric. Res. 1978, 29, 963-974 reports the presence of D-pinitol in two Desmodium species. But this article is about D. intortum and D. tortuosum, again two botanical species that are different from D. adscendens.

The same is true for Database CA (Chemical Abstracts), which refers to a Chinese journal (Zhongcaoyao 2007, 38 (8), 1157-1159), and reports the presence of pinitol in D. microphyllum, which is still further a type which is botanically different from D. adscendens.

The same applies again for Database NAPRALERT, which refers to an Indian magazine (Curr. Sci. 1982, 51, 936-937) and reports the presence of (+)-pinitol, i.e. D-pinitol, in Desmodium triflorum. However, this article deals with D. triflorum, which is again another kind that is botanically different from D. adscendens.

In summary, it can be stated that the fact that other Desmodium species, different from D. adscendens, appear to contain D-pinitol or pinitol, has no predictive value on the presence or absence of this product in D. adscendens however. Two different species belonging to the same genus, always have a different chemical profile of secondary metabolites, both qualitatively and quantitatively. There are numerous plant species belonging to other genera and other plant families, which also contain D-pinitol. In the general reference ‘Dictionary of Natural Products on DVD’, it is explicitly stated “widely distributed in plants” about D-pinitol. This thus implies that there is no consistent or predictable relationship between the genus Desmodium and D-pinitol.

One distinguishes the Desmodium preparation including pinitol as “whole” or totum, with regard to the above documents, which yet mention the presence of pinitol in other Desmodium species, which does not mean although that the occurrence of pinitol in Desmodium adscendens can be expected, on the contrary. That seems all too short-sighted. Indeed, the fact that other Desmodium species, which are different from D. adscendens, appear to contain pinitol, has no predictive value on the presence or absence of this product pinitol in D. adscendens. It is generally known that the medicinal properties of a particular plant species are very often exclusively limited to said only one kind, and these are not present in other species of the same genus and/or family. It is indeed often the case that only a particular plant from a whole plant family is useful for achieving a certain effect, like in this case, D.A. from Desmodium family is mainly intended against certain diseases as further specified, or also like Papaver somniferum versus morphine.

Finally, the publication Short Communication NARAYANAN still further relates to pinitol. But it does not address D. adscendens but Bougainvillea spectabilis.

According to an advantageous embodiment of the invention, a validated analytical method is provided for the quantitation of D-pinitol, wherein a specific quantified D. adscendens preparation is produced as “totum”.

According to a particular embodiment of the invention, an analytical method is developed for obtaining a preparation quantified for D-pinitol, wherein biologically controlled isolation is proposed, in which the product is repeatedly enriched and numerous fractions are isolated, and wherein the decoction obtained also contains a large amount of D-pinitol. Thus because of this biologically controlled isolation according to the invention, the desired constituent in the plant selected in particular from the general plant family can be separated in a more targeted way, which offers the enormous advantage of saving an extraordinarily large volume of research, namely to find this plant component with a desired effect.

According to a particularly advantageous embodiment of the invention, the constituents are standardised. As a result of the natural variation in the plants, both of tropical and of cultivated origin, with respect to the constituents, it is necessary to guarantee the reproducibility and consequently to standardise the constituents.

According to a privileged embodiment of the invention, sugar alcohols, more in particular xylitol, are selected as the internal standard. Actually, an internal standard must always be added in gas chromatography to correct for the variability of the injection. These selected substances have a behaviour that is similar to that of the substance being analysed, in this case D-pinitol. It is thus also possible to choose other sugar alcohols such as sorbitol, mannitol, dulcitol and inositol, besides the above-mentioned xylitol, but the last two mentioned are less often selected. Preference is given to xylitol for a structural reason: xylitol has the same number of hydroxyl groups as D-pinitol, which in this case is a decisive factor, since the derivatisation reaction takes place on the hydroxyl groups.

Using a suitable analytical method, the main components in Desmodium adscendens and the total amount of D-pinitol are determined. The extraction process, the products obtained after extraction and the conditions are evaluated and optimised.

According to a limited embodiment of the invention, in a first instance, above-ground plant parts of D. adscendens are used, especially those less or not at all affected by the season. The result is that harvesting these offers the advantage of providing availability year-around and it also has been experimentally established and implemented.

According to an additional embodiment of the invention, said plant extract is prepared likewise from more temporary above-ground plant parts of D. adscendens, which are more season-dependent, such as blossoms and fruits, and even seeds. It is thus possible to simplify the harvesting of the plant without having to dig up the plant.

According to yet a further additional embodiment of the invention, below-ground plant parts of D.A., which are actually more season-independent, can also be used for preparing said plant extract. Thus almost all plant parts of said Desmodium adscendens can be used, in particular leaves, stems, branches and/or other above-ground parts, but also below-ground parts thereof, and if desired, also blossoms and fruits, or even the seeds thereof.

Thus this offers the great advantage that almost all parts of the plant can be used, so that almost nothing is wasted. This then provides a financially profitable method that is also completely environmentally responsible, which fits perfectly into today's guiding concept of sustainable economy, which is thus particularly significant for the relevant tropical and subtropical areas where this is a decisive criterion.

According to an additional phase of a basic embodiment of the method of the invention, said plant parts of D. adscendens are first dried.

According to a preferred embodiment of the invention, said fraction of Desmodium adscendens is first ground, in particular into powdered form. This powder can optionally be converted directly to a pharmaceutical form.

According to a more preferred embodiment of the invention, an aqueous decoction of said leaves and/or branches of Desmodium adscendens is then prepared as a decoction by boiling a certain quantity of dried, optionally powdered, leaves in a certain quantity of water, in particular distilled water, for a certain amount of time, especially 5×200 g, 3 L, and 1 hour, respectively.

According to a more particularly preferred embodiment of the invention, said aqueous decoction of the Desmodium adscendens plant parts used, such as leaves or branches, is then cooled; still more particularly wherein the portions obtained are then combined and filtered, after which the filtrate obtained is concentrated, especially under vacuum, and then freeze-dried or spray-dried.

According to an even more preferred embodiment of the invention, an extract of said above-ground and/or below-ground parts of Desmodium adscendens is produced with the aid of solvents such as but not exclusively water, lower alkyl alcohols, non-polar solvents or combinations thereof and solvents under supercritical conditions.

In addition to water, as a decoction or macerate, i.e. warm or cold, lower alkyl alcohols may also be used, such as but not exclusively methanol, ethanol and isopropanol or water-alcohol combinations thereof and less polar to non-polar solvents, such as but not exclusively ethyl acetate and n-hexane or combinations thereof. Optionally, solvents under supercritical conditions, such as but not exclusively carbon dioxide, may also be used. Finally, the dried powder of the plant material may also be used without further extraction as an alternative to an extract.

According to a particular embodiment of the invention, from 60 to 70 g, in particular about 65 g, of dried decoction may be obtained starting from 1 kg of dried leaves.

According to a yet more advantageous embodiment of the invention, a certain quantity, in particular about 20 g, of said decoction is subjected to column chromatography, in particular Sephadex LH20 with methanol elution, after which certain fractions, in particular 100 ml, are collected and analysed by thin-layer chromatography, in particular silica gel, layer thickness 0.25 cm, MeOH/H₂O: 5:1 as the mobile phase, and their chromatographic pattern determined.

According to a further embodiment of the invention, said fractions are combined into a number of sub-fractions, in particular 11, according to their chromatographic pattern.

According to yet a further embodiment of the invention, some of the selected said fractions 5-11, in particular 200 mg, more particularly those that show a coloured spot, are combined and these fractions are then subjected to further column chromatography, in particular on Sephadex LH-20 eluted with MeOH, whereupon once again certain fractions, in particular of 100 ml, are collected and analysed.

According to a still further embodiment of the invention, said sub-fractions 4-5, in particular 120 mg, from this column are combined, whereupon these materials are subjected to further column chromatography under the same conditions, after which a pure product is obtained, in particular white.

According to an invention's privileged embodiment, said extract is separated & the isolated product identified as methylated cyclitol 3-O-methyl-chiro-inositol, also called (+)-D-pinitol or D-pinitol.

In addition, according to a further embodiment of this invention with the related data applied to it, an extended characterization of the D. adscendens preparation is possible according to a basic embodiment of this invention with a flavonoid profile.

In addition, MUANDA reports as the most important phenolic component “quercetin dihydrate”, whereas in Table 1 the terms “quercetin glucosyl” and “quercetin dihydrate” are used. The presence of vitexin and derivatives thereof is also not reported.

According to a particular embodiment of the invention, a standardised extract is derived from the plant Desmodium adscendens, quantified for D-pinitol in terms of its effect on hepatitis, in particular preventive, with the separation of D-pinitol from Desmodium adscendens, wherein D-pinitol forms an active constituent, in particular regarding liver injury of chemical, physical, infectious or immunologic origin.

In contrast, however, document WO 2004/084875 relates to pinitol, or a pinitol-containing plant extract for the protection of the liver. This document also reports as plants soybean, pine, Hovenia dulcis, Acanthopanax senticosus and carob, but does not discuss Desmodium, and thus even less D. adscendens. Furthermore, this document describes SOD and glutathione as indicators for the liver-protective effect. So there is no description here of the use of an extract from a D-pinitol containing plant for protecting liver function, which indicates any method for preventing any liver disorder, even less treating same, and certainly not an exotic, i.e., tropical plant with the use of D-pinitol or any extract containing this component.

Furthermore, it relates to hypoglycaemic and anti-diabetic activity, but in no way to a hepato-protective effect, neither to the treatment of a liver disorder.

This invention also relates to a plant extract obtained according to a method as described above for use in protecting the liver of a mammal, i.e., for preventing a liver disorder therein.

The presence of pinitol in other Desmodium species surely does not detract from the finding that a D. adscendens preparation characterised by its content of pinitol has a hepato-protective effect, given that this is not the case for said other Desmodium species. In terms of the invention, however, it is true that this allows the preparation of a standardised extract derived from a known plant, Desmodium adscendens, quantified for the D-pinitol molecule in its action against hepatitis. The point is that the extraction of D-pinitol from Desmodium adscendens discussed here is in no way known, since the related plant Desmodium adscendens is used for numerous purposes, even many others than those mentioned here. The scope of the solution which is aimed for, however, lies in the deliberate and targeted selection of a certain indication thereof. There are references yet to numerous products including amino acids, etc., but there is no mention at all of D-pinitol, which is clearly responsible for the activity in the specific plant D. adscendens against liver disorders, nor is this suggested, and with the additional advantage that it is present herein in considerable amounts, as a principal constituent thereof, which can furthermore be suitably separated according to the invention.

According to an additional embodiment of the invention, said plant extract is used for treating a liver disorder in a mammal, in particular one resulting from chemical causes or caused by infections, in particular by bacteria or viruses, as well as physical or immunologic causes.

According to a particular embodiment of the invention, the D-pinitol-containing plant extract is administered to the mammal in the form of a composition containing it, wherein said composition is selected from the group consisting of a pharmaceutical composition, a food composition and/or a beverage composition, with reference to an anti-hepatotoxic activity of a quantified Desmodium adscendens decoction and D-pinitol against chemically-induced liver injury, particularly in the sector of ethno-pharmacology.

Additional features and particularities of the method according to the invention are defined in the additional sub-claims. Further details are incorporated in the following description of some preferred exemplary embodiments of the method according to the invention, which is illustrated based on the attached drawings.

### BRIEF DESCRIPTION OF THE DRAWINGS

FIG. 1 shows a functional working diagram of the method according to the invention in the form of a flow chart;

FIG. 2 is a realistic reproduction of a representative sample of leaves and blossoms of Desmodium adscendens;

FIG. 3 is a schematic representation of insulin signal transduction;

FIG. 4 is a graphical representation of the calibration curve obtained after derivatisation with BSTFA;

FIGS. 5 to 7 are each graphical representations of concentration and area ratios after 3 h and after 6 h, respectively, of derivatisation and after overnight derivatisation;

FIGS. 8 and 9 are each graphical representations of the concentration and area ratios after 1 h of derivatisation in a heat block;

FIGS. 10 and 11 are each a graphical representation of the area ratio of D-pinitol/internal standard for 100 mg sample for extraction in an ultrasonic vibration bath;

FIGS. 12 and 13 are each a graphical representation of the D-pinitol/internal standard area ratio per 100 mg sample for extraction in a heated vibrating bath and by extraction/reflux;

FIG. 14 is a graphical representation of the mean area ratios for the various extraction methods;

FIG. 15 is a graphical representation of a response function;

FIG. 16 is a graphical representation with interferences which gives an overview of residues;

FIGS. 17 and 18 are each graphical representations of individual measurements and mean values on various days;

FIGS. 19 and 20 are each graphical representations of values recovered after use of the so-called standard addition method;

FIGS. 21 and 22 are each a partial chromatogram of analyses respectively without and with internal standard showing the voltage in mV versus minutes;

FIGS. 23 and 24 are each complete chromatograms of the analysis of the standards showing the voltage in mV versus minutes;

FIGS. 25 to 28 are each a mass spectrum for various substances and xylitol standards showing the relative influence in percent versus the most intense signal in terms of specificity;

FIGS. 29 to 31 show serum AST values 48 h after acetaminophen administration, and serum ALT values 48 and 72 h after acetaminophen administration;

FIGS. 32 to 38 analogously show additional experimental data, with FIGS. 35, 39 showing the further UV spectrum of peaks A-F, FIG. 40 the graphical survival percentage and FIG. 41 an additional chromatographic profile.

### DESCRIPTION

This invention generally relates to a method in which the various steps are described schematically and which is used for preparing a special plant extract from Desmodium adscendens, the above-ground and/or below-ground parts of which serve as the starting product in this production process. Desmodium adscendens is a herb that belongs to the family of the Fabaceae and the genus Desmodium, a fragment of which is shown in FIG. 2. This is a hardy plant that can grow 0.5-1 m tall and has a round, hairy, vining stem with grooves. The plant is 3-leaved. The supporting leaflets are hairy to hairless at the outer edge and are 0.5-1 mm long and 1.5-3 mm wide. They are winter-hardy. The leaf stalk is hairy and 1-3 cm long. The leaves are elliptical—inverted egg-shaped, blunt and scalloped at the top and wedge-shaped-round at the base. The leaves are primarily hairless on the top and very hairy on the underside.

The manner of blossoming consists of axial and terminal clusters. The leaf stalk is grooved and profusely hairy to fine-haired. The initial bracts are oval-sharp pointed with a pointed top and 3.5-5 mm long and 1.5-2 mm wide. The blossom stalks have the same hair pattern as the leaf stalks and are 0.4-1.7 cm long. The petals are mostly in pairs. The flower crown is larger than the calyx and is oval. The fruit has extended peduncles of 0.5-2 mm long and is 1-5 membered, obliquely elongated with a dimension of 3.5-5.5 mm×2.5-3 mm. The seed is transversely elliptical and 2.5-5 mm long and 1.5 mm wide.

Desmodium adscendens has several pharmacologic properties. In the neuro-pharmacologic area an extract of the plant has a depressive activity on the central nervous system. The ethanolic extract has analgesic and hypothermic activity and inhibits the propagation of tonic-clonic convulsions.

Aqueous and ethanolic extracts of this plant reduce smooth muscle contractions and reduce the release of substances that activate smooth muscle, cells in the lungs. Various fractions of the extract are being studied. One sub-fraction inhibits smooth muscle cell contraction induced by antigen via inhibition of phospholipases, which occurs because of activation of calcium-activated potassium receptors. Saponins are present in this fraction. Furthermore, the fraction that contains a tetrahydroisoquinoline analogue inhibits the cytochrome P450 NADPH-dependent mono-oxygenase reaction which produces epoxy- and hydroxyeicosanoids. The fraction increases the COX activity, which results in increased prostaglandin production.

In the experimental phase, first a number of tests were conducted in vitro, and then additional ones in vivo. The sequence of the test described in the following is shown schematically in the flow chart of FIG. 1.

In a phytochemical study of Desmodium adscendens plant material and extraction thereof from Ghana delivered an aqueous decoction of the leaves, which was prepared by boiling 5×200 g of dried and pulverized leaves in 3 l of distilled water for 1 hour. After cooling, the portions were combined and filtered. The filtrate was concentrated under vacuum and then lyophilized. Starting from 1 kg of dried leaves, approximately 65 g dry decoct was typically achieved.

Then, 20 g of decoct subjected to column chromatography on Sephadex LH20 (120×4 cm) with methanol elution. Fractions of 100 ml were collected and analyzed by thin layer chromatography (silica gel Merck, layer thickness 0.25 cm, MeOH/H2O: 5:1 as mobile phase). Spots were detected under UV light, in particular below a wavelength of 366 nm. After spraying with 1% anisaldehyde/H2SO4 in MeOH, the plate was heated to 120° C. for 10 min in order to obtain colored spots. Fractions were combined in 11 sub-fractions according to their chromatographic pattern. Subfractions 5-11 (200 mg) showed a spot with a green color, and were pooled. This fraction was subjected to a further column chromatography on Sephadex LH20 (60×3 cm) eluted with MeOH, and fractions of 100 ml were collected again and analyzed as described. Sub-fractions 4-5 (120 mg) from this column were combined, and after a new column chromatography under the same conditions, a crystalline product was obtained.

The phase of structural clarification by spectroscopic examination with ¹H, ¹³C NMR and mass spectroscopy and measurement of the specific optical rotation led to identification of the isolated product as the methylated cyclitol 3-O-methyl-chiro-inositol, also known as (+)-pinitol or D-pinitol.

Treatment of 3T3-L1 adipocytes with 0.5 and 1 mM D-pinitol increases the mRNA expression of glucose transporter (GLUT4), insulin receptor substrate (IRS), peroxisome proliferator activated receptor γ (PPARγ) and CCAAT/enhancer-binding proteins (C/EBP). 1 mM D-pinitol increases expression of adiponectin mRNA, an adipocytokine with anti-inflammatory, anti-diabetic and anti-atherogenic properties, the expression of which is also increased by insulin. The increased expression of a number of factors can be explained by the insulin mimetic properties of D-pinitol.

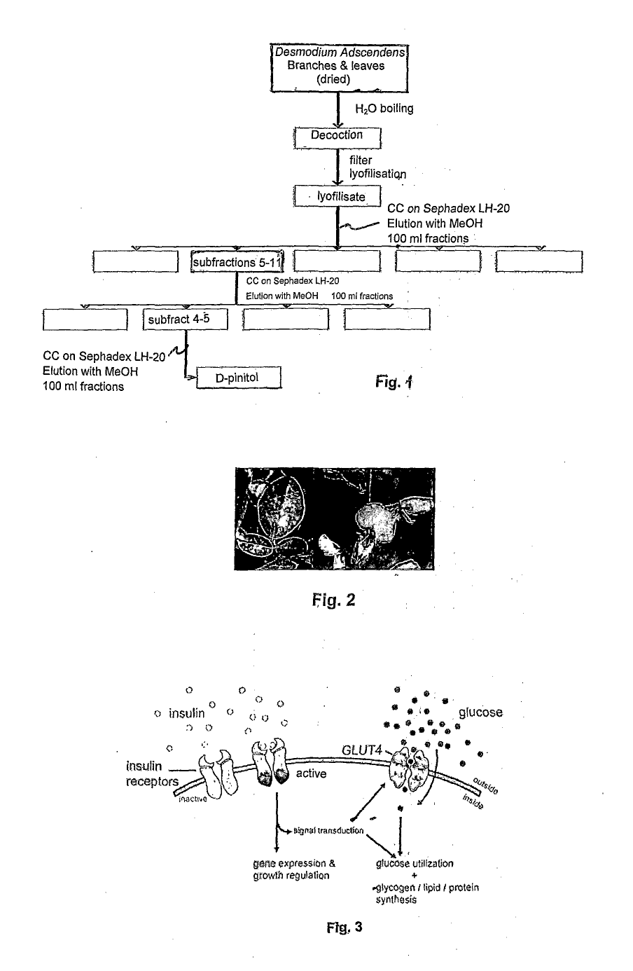

In L6 rat muscle cells, D-pinitol induces the translocation of GLUT4 to the cell membrane, like insulin, and readies it for the uptake of glucose, see FIG. 3.

With regard to anti-atherogenic activity it was found that D-pinitol moderately decreases the formation of foam cells by reducing the secretion and expression of cytokines such as TNF-α, monocyte chemoattractant protein-1, IL-1beta and IL-8 and reducing the expression of macrophage scavenger receptor, CD36 and CD86. The insulin mimetic activity of D-pinitol is probably responsible for this.

Regarding the effect on the immune system, it was found D-pinitol has immunopharmacologic properties and that D-pinitol decreases the expression of MHC-I, MHC-II and co-stimulators such as CD80 and CD86, both in vitro and in vivo, by suppressing MAPKs activation and translocation of NF-kB, and reduces the production of large quantities of IL-12 and pro-inflammatory cytokines in LPS-induced dendritic bone marrow cells. This results in the inhibition of maturation of these cells. Treatment of dendritic cells with D-pinitol prevents these cells from inducing a normal cell-mediated immune response, and when LPS-stimulated dendritic cells are treated with D-pinitol, the proliferation of T-cells and the production of INF-γ by CD4+ cells are affected negatively. In neutrophils, D-pinitol inhibits TNF-alpha expression.

D-pinitol inhibits constitutive and induced NF-kB activation in a dose- and time-dependent manner. The inhibition is not cell-specific and takes place through inhibition of IKK activation, IkBa degradation and phosphorylation, nuclear phosphorylation and translocation of p65. D-pinitol also reduces NF-kB-dependent reporter gene expression and suppresses NF-kB-dependent gene products involved in cell proliferation, anti-apoptosis, invasion and angiogenesis. This can explain why analogues of D-pinitol, such as azole nucleoside analogues, have anti-tumour properties. Other derivatives of D-pinitol such as aminocyclitols inhibit glycosidase.

In vivo pharmacologic properties were studied in test animals. In the tests conducted in vivo, the liver was injured and the preventive and/or curative effects were studied. Until that time it had not been proven that the molecule involved had curative action, although the preventive character thereof was well-proven. In addition to activity in diabetic mice and rats, in streptozotocin-induced diabetic mice—in which D-pinitol has an acute and chronic hypoglycaemic effect—it increases the basal uptake of 2-deoxyglucose in L6 muscle cells by intervening in insulin signal transduction. D-pinitol is not effective in severely insulin-resistant mice. In streptozotocin-induced diabetic rats, D-pinitol lowers blood glucose haemoglobin and increases insulin, while D-pinitol also normalises aspartate transaminase (AST), alanine transaminase (ALT) and alkaline phosphatase values in the liver and has a lipid-lowering effect. The antioxidant effect is manifested in the reduction of lipid peroxidation and hydroperoxidation, an increase in non-enzymatic antioxidants and normalization of the enzymatic antioxidants superoxide dismutase (SOD), glutathione peroxidase (GP), catalase and glutathione-S-transferase (GST).

As far as the hepato-protective effect is concerned, the substance normalises aspartate transaminase (AST) and alanine transaminase (ALT) liver values and TNF-α values after induction of liver injury with galactosamine. In addition, D-pinitol reduces lipid peroxidation and normalises the glutathione, glutathione reductase and glutathione peroxidase values.

It also has an anti-inflammatory effect: in rat studies D-pinitol had anti-inflammatory properties, both against acute (carrageenan-induced oedema of the paw) and subacute (cotton pellet granuloma) inflammation. The substance also has anthelmintic activity.

With regard to the effect on the immune system, in mice with OVA-induced asthma, D-pinitol decreases the number of inflammatory cells in bronchoalveolar lavage fluid and reduces the infiltration of these cells into peribronchiolar and perivascular regions. The inflammation in the lungs is thus combatted. The Th2 cytokines such as IL-4, IL-5 and eotaxins decrease through intake of D-pinitol and the Th1 cytokines such as INF-γ increase, as do the INF-γ positive CD4 cells. The Th1/Th2 balance is also corrected by D-pinitol through increased expression of the Th1 transcription factor T-bet and a decrease in the transcription factor GATA-3, which is elevated in Th2 pathologies. The gelatinolytic activity of MMP-9 in lung tissue, which is important in the migration of inflammatory cells from blood to tissue, is decreased. D-pinitol thus reduces the hyperreactivity and inflammation observed in asthma.

Studies in Humans

In patients with type 2 diabetes, D-pinitol has a favourable effect on fasting glucose values, HbA1c values and insulin. Total cholesterol, LDL/HDL ratio and systolic and diastolic blood pressures decreased after 13 weeks of treatment with 2×60 mg D-pinitol per day, while the HDL cholesterol values increased. In patients with uncontrolled type 2 diabetes, 12 weeks of treatment with 20 mg/kg/day of D-pinitol, in addition to the usual therapy, improves the fasting and postprandial glucose values as well as HbA1c values, but does not significantly change the levels of adiponectin, leptin, C-reactive protein (CRP) and free fatty acids. When D-pinitol is taken at a dosage of 20 mg/kg/day for a shorter period of 4 weeks, the substance has no effect on basal and insulin-mediated glucose or lipid metabolism in insulin-resistant patients. In older, non-diabetic patients, D-pinitol intake for 6 weeks has no effect on insulin-mediated glucose metabolism.

The development of an analytical method for D-pinitol in D. adscendens decoction is presented in the following with the corresponding validation. Before a standardised preparation can be produced from plant material, the concentration of the active substance in the plant must be known. For this purpose a standard method which is replicable, accurate, not time-consuming and also preferably economical is used. The goal of this master study is the development of an analytical method for determining the content of D-pinitol in a decoction of D. adscendens. Various parameters were investigated for this purpose, such as the analytical technique (GC, HPLC, TLC), the column, the detector and the optional purification. Once a possible method is developed, it should also be validated via the ICH standards. Thus the linearity, range, reproducibility, accuracy and specificity will be investigated.

Materials selected included methanol, HPLC grade with analytical quality, pyridine (99+%), BSTFA 1% TMCS, standards D-pinitol (95%), xylitol (99% minimum) and dulcitol (99+%), mannitol p.a. and sorbitol, helium, hydrogen gas and air, sodium chloride (p.a.), D(+)-galactosamine hydrochloride (99% minimum) and silymarin.

The decoction of Desmodium adscendens was prepared in the following way: 3 litres of distilled water were added to 200 g plant material. The entire product was boiled for 1 h, after which it was cooled and filtered. Volume reduction was accomplished by evaporation using a Rotavapor. As the last step, the decoction was freeze-dried.

In the thin-layer chromatography technique, the stationary phase is on a plastic or glass support. The stationary phase can be silica gel or modified silica, but also aluminium oxide, cellulose or diatomaceous earth.

1-10 μl of the solution to be analysed is spotted at approximately 1.5 cm from the bottom edge of the plate. Then the plate is placed in a developing tank containing a layer of about 1 cm mobile phase. The mobile phase rises by capillary force and, depending on the type of substances in the solution, the substances elute rapidly or slowly. When the liquid front has almost reached the top edge of the plate, this is removed from the developing tank and the mobile phase evaporated. It is then possible to visualise a spot pattern using UV light or treating with spray reagents and calculate the retention times. A qualitative determination can be performed using a densitometer, which measures the intensity of the spots and converts this into a densitogram.

For the experiments, silica gel F₂₅₄ silica gel TLC-plates, Lichrospher silica gel F₂₅₄ HPTLC-plates and silica gel 60 RP-18 F₂₅₄ plates were used.

Gas chromatography GC is an analytical technique wherein analytes are separated by partitioning between the stationary liquid phase and a mobile gas phase. Because of the high temperature in the injection block, during the injection both the solvent and the analytes evaporate and condense on the cooler column. When the column temperature is increased, the relatively less volatile analytes also enter the gas phase and finally reach the detector. The more non-polar and more volatile the analyte is, the more affinity it has for the gas phase and the shorter is its retention time. More polar or less volatile analytes have more affinity for the more polar stationary phase and reach the detector later. Possible detectors are the flame ionisation detector, electron capture detector, nitrogen-phosphorus detector, katharometer, sulphur detector and mass spectrometer.

For these experiments a GC-FID of the type Trace GC Ultra with FID detector was used. To check the specificity, a GC-MS of the type Trace 2000 GC with a Voyager EI MS detector was used.

A flame ionisation detector (FID) and a mass spectrometer (MS) are used in these experiments. The flame ionisation detector is a universal and sensitive detector. The eluate is mixed with H₂ and air, and burned. As a result, organic compounds ionise, and the ions increase the current strength relative to a collector electrode at constant voltage. The mass spectrometer works by ionisation of molecules, after which the ions are measured.

Liquid chromatography is an additional analytical technique in which the separation takes place through a difference in distribution between the stationary phase and the liquid mobile phase. If the stationary phase is more polar than the mobile phase, the term “normal phase chromatography” is used, while if the stationary phase is non-polar, “reversed phase chromatography” is the term applied. The analytes are eluted with eluent, the composition of which, and thus also the polarity, can be modified to elute analytes more quickly or more slowly. Possible detectors are UV detectors, RI detectors, amperometric detectors, mass spectrometers and evaporative light scattering detectors (ELSD).

An ELSD was used for these experiments, wherein after evaporation of the mobile phase, the light scattering was measured and converted to an electrical signal.

Possible analytical methods for the analysis of sugars and sugar derivatives, including sugar alcohols such as cyclitols, are known. Several techniques are available for the determination, and a brief overview of these is given below.

For example, for GC techniques, since sugars and sugar derivatives are not volatile, a derivatisation step must also be added here. Both trimethylsilylation and acetylation are known. Both the general flame ionisation detector and the mass spectrometer can be used as detector.

Further, HPLC techniques: since the analytes are not UV-active, they must be derivatised before UV detection is possible. UV-active substances are prepared by, among other things, derivatisation of benzyl chloride or by means of UV-active ion pair reagents. In addition to UV detection, refractive index detection, pulse amperometric detection, mass spectrometry or electron light scattering detection without derivatisation may also be used. Both C₁₈-columns and anion exchange columns can be used. If anion exchange columns are used, the sugars are converted to anions with the aid of a base.

Other possible techniques are enzymatic assays, capillary electrophoresis and thin-layer chromatography-densitometry. In thin-layer chromatography as described by Pothier, based on fingerprinting by means thereof, attempts were also made to separate D-pinitol from the other substances in the decoction, since TLC methods are quick and relatively inexpensive.

D-pinitol (standard) and the sample were dissolved in distilled water and 50% methanol-50% water. The mobile phases used were ethyl acetate-formic acid-acetic acid-water (67.5:7.5:7.5:17.5), chloroform-methanol-water (54.5:36.5:9), chloroform-methanol-water (55:36:9), chloroform-methanol-water (46.5:46.5:7) & chloroform-methanol-water (33:53.5:13.5).

D-pinitol was not visible under UV light on the plates with the UV indicator F₂₅₄ₛ, not even at very high concentrations (10 mg/ml), while several spots were seen for the standard when anisaldehyde or thymol was used as the spray reagent.

Therefore the decision was made to develop a method using a different analytical technique. Since sufficient GC methods are described in the literature for analysing D-pinitol, and gas chromatography is a more sensitive technique than that with HPLC derivatisation, the decision was made to develop a GC method.

Based on the determination of D-chiro-inositol in buckwheat using HPLC-ELSD, an attempt was made to develop a liquid chromatographic method in parallel with the gas chromatographic method. Initial experiments indicated that the reproducibility was very low. Therefore this method was not further optimised.

Therefore an immediate change to gas chromatography was made. It is known that various columns can be used for analysis of sugars and sugar derivatives. In the Table below, an overview is given of some of these columns.

TABLE 1
alkylsilicones

| | | |
|---|---|---|
| Non polar simplicity | 100% methylpolysiloxane | Inositols and methyl-in H. rhamnoides |

alkylphenylsilicones

| | | |
|---|---|---|
| DB-5MS | 5% phenyl polysilphenylene siloxane | Sugars in environmental samples |
| SPB-20 | 20% phenyl 80% methylpolysiloxane | Inositol isomers and arabitol in urine |
| ID.BPx5 | 5% phenyl 95% methylpolysiloxane | Sugars in leaves of Forsythia spp. |

cyanoalkylsilicones

| | | |
|---|---|---|
| SPB-1701 | 14% cyanopropylphenyl 84% methylsiloxane | Polyols in urine |
| DB-225 | 50% cyanopropylphenyl 50% methylsiloxane | Carbohydrates in D. microcarpum |

packed columns

| | |
|---|---|
| OV-17 | Pinitol in soybeans |
| OV-17 (3%) | Carbohydrates in soybeans |

Columns DB-5 and HP-5 (5% phenyl 95% methylpolysiloxane) and AT™—5MS/RTX-5MS (5% phenyl polysilphenylene siloxane), of the Alltech type, which are moderately polar, and the column AT™—1/RTX-1 (100% methylpolysiloxane) of the Restek type, which is a column with a non-polar stationary phase, were used. Since a decoction is obtained by boiling plant material in water and thus principally contains polar substances, it was expected that the analytes would have more retention on the 5% phenyl 95% methylpolysiloxane column than the 100% methylpolysiloxane column, and that this would give better separation. Therefore the HP-5 column was chosen over the AT™-1/RTX-1 column for analysis of the decoction. A column with cyanoalkylsilicones as the stationary phase is the column AT™-264.6% cyanopropylphenyl and 94% methylsiloxane.

An internal standard must also be determined. Since gas chromatography is used here, an internal standard must be added to correct for variations in the injection. An internal standard must behave similarly to the substance being analysed. Therefore it was chosen to use some available substances of structures similar to that of D-pinitol, namely sugar alcohols such as sorbitol, mannitol, dulcitol, xylitol and inositol, which are shown in the following.

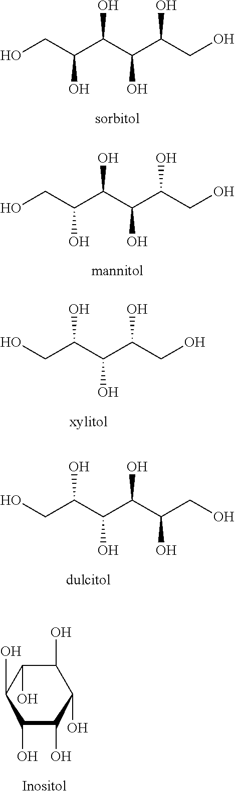

Monosaccharides such as lactose, however, were not selected since they gave two peaks in the chromatogram as a result of anomerization. This increases the chance of interference with the internal standard; in other words, there is a greater chance that peaks of the sample will overlap with those of the internal standard. In the case of disaccharides, there is always a chance of breakdown, which is difficult to monitor and is undesirable for quantitative analysis. Sugars or sugar derivatives with a high molecular weight compared to D-pinitol elute at a late time and thus extend the duration of the analysis, and therefore this compound was also not selected.

Of the five sugar alcohols selected, dulcitol and inositol are poorly soluble in methanol, but are soluble in water. These substances are not the first choice, since water, even in trace amounts, can cause degradation of the TMS derivatives, which does not help with quantitative analysis. Another advantage is that water takes longer to evaporate to dryness than methanol. Furthermore, very small amounts of water, i.e. trace amounts, are not visible to the naked eye and a drying agent should be used. The influence of this substance on analysis then must also be determined.

Mannitol, sorbitol and xylitol are soluble in methanol, but the retention times of mannitol and sorbitol overlap with those of another unknown in the sample, as is apparent from Table 2 below. Xylitol elutes at a time when no other substance elutes in the chromatogram and only noise is apparent. Structurally also, xylitol is a better choice than mannitol or sorbitol, since xylitol contains the same number of hydroxyl groups as D-pinitol. This is important, since the derivatisation reaction takes place on the hydroxyl groups. Table 2 below contains an overview of the retention times of the possible internal standards.

TABLE 2

Retention
time

| | | |
|---|---|---|
| Dulcitol | 24.66 | |
| Inositol | 24.96 | 26.82 |
| Mannitol | 24.46 | |
| Sorbitol | 24.57 | |
| Xylitol | 20.77 | |

As far as the derivatisation is concerned, sugar derivatives can be derivatised to volatile derivatives by acetylation or silylation. Various reagents have been used in the literature, including BSTFA+TMCS in pyridine, HMDS+TFA, HMDS+TMCS in pyridine, STOX+HMDS+TFA, TMSI+ pyridine, acetic anhydride+pyridine, and AcO-N-methylimidazole. Among all these possibilities it was decided to perform acetylation with acetic anhydride and pyridine and trimethylsilylation with BSTFA+1% TMCS (+pyridine) and compare the results.

The acetylation was performed with acetic anhydride and pyridine in a 2:1 ratio. The derivatisation mixture was added to the weighed quantity of solid, D-pinitol, and sorbitol was added as internal standard. In this process it was necessary to search for the correct volume of derivatisation mixture so that D-pinitol would be soluble in it. The mixture was either heated for 30 minutes in the oven at 60° C., or the vial was stored overnight at room temperature. After derivatisation the samples were evaporated to dryness under a nitrogen stream, after which the derivatives were redissolved in ethyl acetate and analysed. An AT™-264 column was used for this purpose, in analogy to the literature where a DB-225 column was used for analysis of acetylation derivatives (Ac20-N-methylimidazole). The temperature of the oven was 200° C. and was raised at 5° C./minute to 220° C., which was maintained for 20 minutes.

The peaks showed a very great variation with regard to retention time, and the reproducibility of the peak areas left something to be desired. One explanation might be that the derivatives, from a relative viewpoint, are not volatile enough, since 5 hydroxyl groups had to be derivatised.

When trimethylsilylation was used, D-pinitol and sorbitol were weighed out and dissolved in 2 ml methanol. Sorbitol was used for the first derivatisation experiments, and then later the internal standard was chosen as described above. 100 μl were evaporated to dryness under a stream of nitrogen, with 100 microlitres of BSTFA+1% TMSC and 40 μl of pyridine being added. The vial was held for 3 h in an oven at 70° C. Then the derivatisation reagent was evaporated off and the residue was re-dissolved in 300 μl hexane. Each level was weighed once but derivatised in duplicate and injected in triplicate. The reaction of the derivatisation of a substance containing a hydroxyl group with BSTFA is shown below:

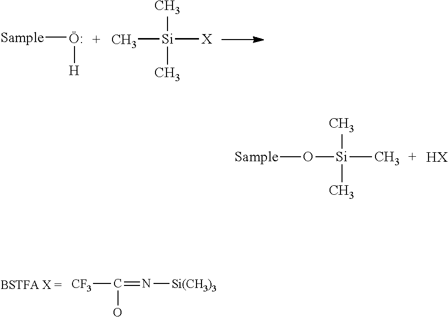

Table 3 below gives an overview of the concentration ratio & the area ratio for D-pinitol/internal standard.

TABLE 3

| Ratio of concentrations | Ratio of areas |
|---|---|
| 0.29 | 0.25 |
| 0.96 | 0.83 |
| 1.74 | 1.38 |

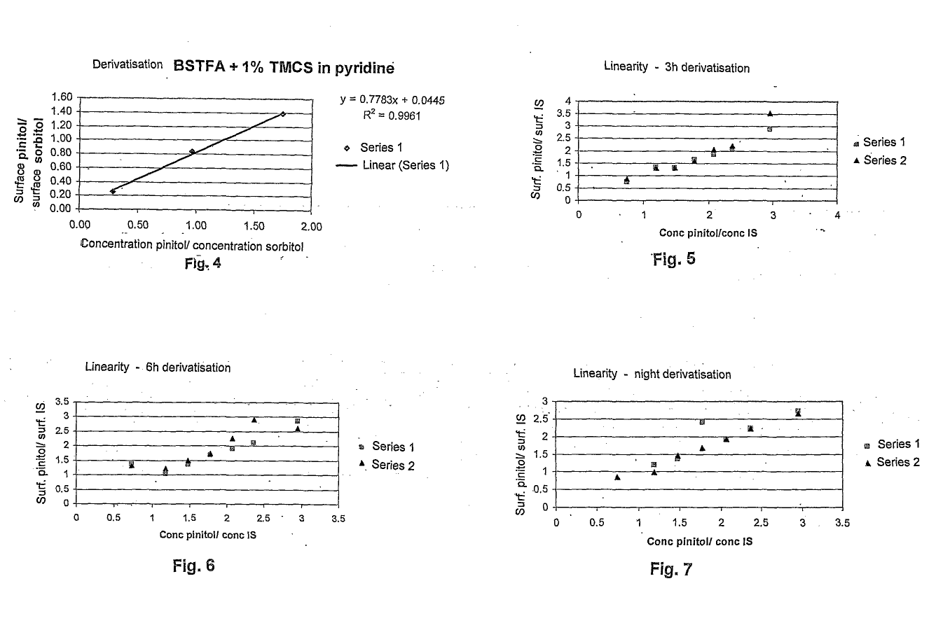

FIG. 4 shows a calibration curve obtained after derivatisation with BSTFA, which appears linear. At first glance this method appears linear and the retention time also remains the same. It was possible to determine from the results of the initial experiments that the derivatisation method is more successful than acetylation. Therefore this derivatisation method was used further. Then a further look was taken at derivatisation with xylitol as the selected internal standard. Furthermore it was determined whether the reaction time causes any change in the areas.

A methanolic solution of D-pinitol and xylitol was prepared with batches of respectively (25 mg/50 ml) and (10 mg/50 ml). In 10 ml volumetric flasks, 2 ml internal standard solution was placed and various quantities of D-pinitol were added. Then these were diluted. 500 μl of this solution was evaporated to dryness under a stream of nitrogen. Then 0.1 ml of derivatisation mixture was added to each vial and the vials were placed in an oven at 70° C. for 3 hours, 6 hours and overnight. Then the derivatisation reagent was evaporated off, the residue re-dissolved in 300 μl hexane and injected into the GC in duplicate.

Table 4 below gives an overview of the concentration ratio and area ratio after three hours of derivatisation, showing linearity.

TABEL 4

| Concentratie | oppervlakte | oppervlakte |
|---|---|---|
| D-pinitol I/IS | D-pinitol I/IS reeks1 | D-pinitol I/IS reeks2 |
| 0.741 | 0.746 | 0.870 |
| 1.185 | 1.324 | 1.292 |
| 1.481 | 1.318 | 1.302 |
| 1.778 | 1.654 | 1.595 |
| 2.074 | 1.862 | 2.059 |
| 2.370 | 2.084 | 2.198 |
| 2.951 | 2.892 | 3.550 |

TABEL 5

| Concentratie | oppervlakte | oppervlakte |
|---|---|---|
| D-pinitol I/IS | D-pinitol I/IS reeks1 | D-pinitol I/IS reeks2 |
| 0.741 | 1.374 | 1.303 |
| 1.185 | 1.041 | 1.212 |
| 1.481 | 1.356 | 1.498 |
| 1.778 | 1.676 | 1.723 |
| 2.074 | 1.905 | 2.263 |
| 2.370 | 2.106 | 2.920 |
| 2.951 | 2.849 | 2.599 |

FIG. 5. is a graphical representation of the concentration & area ratios after 3 hours of derivatisation.

Table 5 above gives an overview of the concentration and area ratios after 6 hours of derivatisation, while FIG. 6 is a graphical representation of the concentration and area ratios after 6 hours of derivatisation.

Table 6 below gives an overview of the concentration and area ratios after overnight derivatisation, while FIG. 7 is a graphical representation of the concentration and area ratios after overnight derivatisation.

TABLE 6

| Concentration of | Area of D-pinitol | Area of D-pinitol |
|---|---|---|
| D-pinitol I/IS | I/IS Series 1 | I/IS Series 2 |
| 0.741 | — | 0.840 |
| 1.185 | 1.200 | 0.977 |
| 1.481 | 1.353 | 1.432 |
| 1.778 | 2.407 | 1.671 |
| 2.074 | 1.905 | 1.916 |
| 2.370 | 2.215 | 2.232 |
| 2.951 | 2.718 | 2.647 |

Regardless of the derivatisation time there is great intervariability for a point with the same concentration, as was apparent from FIGS. 5 to 7 and the 3 tables above. No conclusions could be drawn from this regarding the completeness of the derivatisation reaction. Another problem was that when samples were left in the oven overnight, some of the vials were empty in the morning, and the derivatisation reagent had apparently evaporated. This is also not desirable for a quantitative measurement. For practical purposes it is extremely difficult to allow samples to derivatise for 6 hours.

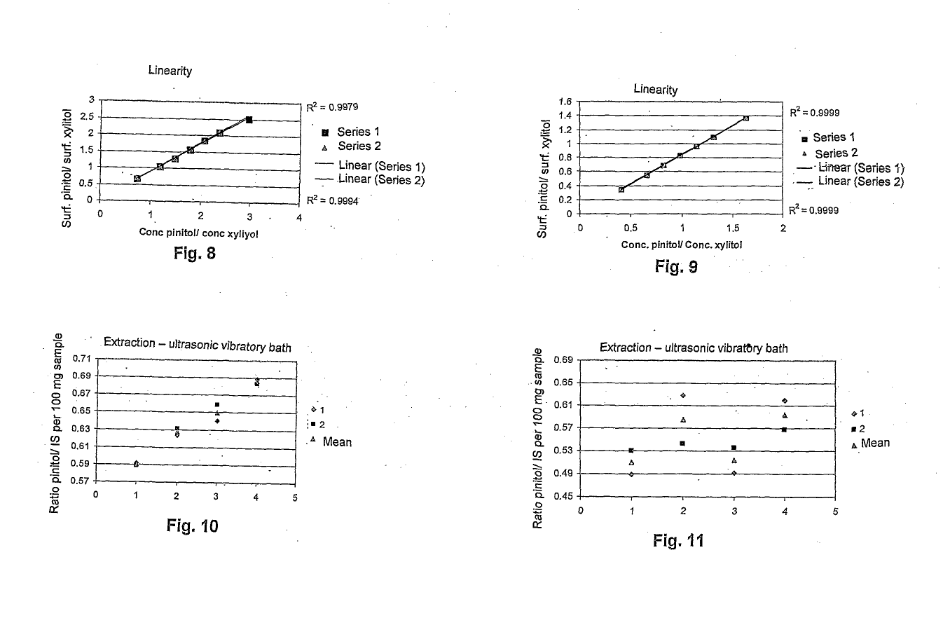

Then the choice was made to use a heating block to prevent this. Reaction vials, which conducted the heat better, were also used. One hour of derivatisation should be adequate with this method. From a solution of D-pinitol and xylitol in respective quantities of 12.5 mg/100 ml and 10 mg/50 ml, 2 ml internal standards and different quantities of D-pinitol were pipetted into 10 ml volumetric flasks and diluted. 50 μl of this solution was evaporated to dryness under a nitrogen stream. 50 μl of derivatisation mixture was added to each vial and the vials were placed in the heating block for 1 hour at 70° C. Then 100 μl of hexane were added, vortexed and injected into the GC in duplicate. Table 7 below shows an overview of the concentration and area ratios after 1 hour of derivatisation in a heating block, while FIG. 8 is a graphical representation thereof.

| Concentration | Surface | Surf |
|---|---|---|
| D-pinitol I/IS | D-pinitol I/IS series1 | D-pinitol I/IS |
| 0.741 | 0.658 | 0.681 |
| 1.185 | 1.039 | 1.040 |
| 1.481 | 1.299 | 1.297 |
| 1.778 | 1.559 | 1.567 |
| 2.074 | 1.840 | 1.832 |
| 2.370 | 2.099 | 2.099 |
| 2.951 | 2.508 | — |

| Concentration | Surface | Surface |
|---|---|---|
| D-pinitol I/IS | D-pinitol I/IS ser.1 | D-pinitol I/IS ser.2 |
| 0.408 | 0.345 | 0.349 |
| 0.653 | 0.551 | 0.551 |
| 0.817 | 0.690 | 0.690 |
| 0.980 | 0.825 | 0.826 |
| 1.144 | 0.983 | 0.966 |
| 1.307 | 1.096 | 1.095 |
| 1.634 | 1.365 | 1.368 |

In a subsequent experiment, a higher concentration of xylitol was used and no further hexane was added after the derivatisation reaction was complete. In this way it was possible to avoid an extra step in the analytical procedure. Table 8 above shows an overview of the concentration and area ratios after 1 hour of derivatisation in a heating block.

When the derivatisation reaction is performed in reaction vials and in a thermal heating block, there are no longer any outliers among the points on the calibration curve. Thus there is a clear difference with respect to the use of the oven. Comparing the two graphics it is seen that the addition of hexane has a great influence on the method. Nevertheless, it was chosen to skip this step in order not to use solvent unnecessarily and to make the analysis less labour-intensive.

Now the extraction: to work quantitatively, the extraction of the sample must also be complete. In the following experiment the best extraction method was sought.

In a first experiment, extraction was performed with an ultrasonic vibrating bath. For this purpose in each case approximately 100 mg of sample was weighed out. In this case, 2 ml of internal standard xylitol (18 mg/50 ml) were added and diluted to 10 ml with methanol. 50 μl of this was evaporated to dryness, derivatised and injected into the GC. In the table below an overview is given of the area ratio of D-pinitol/xylitol for extraction 2, 3 or 4 times and when a larger volume is used. Table 9 below shows an overview of area ratio of D-pinitol/internal standard per 100 mg sample after various numbers of extractions on an ultrasonic vibrating bath, while FIG. 10 is a graphical representation thereof, in which 1 represents 2× extraction, 2 represents 2× extraction in 20 ml versus 10 ml, 3 represents 3× extraction and 4 represents 4× extraction, respectively.

TABLE 9

| | 2x extraction | 2x extraction (20 ml) | 3x extraction | 4x extraction |
|---|---|---|---|---|
| 1 | 0.592 | 0.623 | 0.640 | 0.689 |
| 2 | 0.588 | 0.631 | 0.659 | 0.683 |
| mean | 0.590 | 0.627 | 0.650 | 0.686 |

From the above table and FIG. 10, it can thus be concluded that the extraction is not yet complete after two or even three extraction cycles. To find out whether extraction is complete after four cycles, it would be necessary to perform a fifth cycle. This process was too labour-intensive, and therefore further study was conducted on how the substance can best be extracted. A larger volume also plays a role; when the volume is doubled, the area ratio likewise increases.

In the next experiment the samples were dissolved in 50 ml and 100 ml methanol, together with the same amount of internal standard as before. The samples were vibrated for 30 min or 1 hour on the ultrasonic vibrating bath. Then a certain quantity was evaporated to dryness, derivatised and analysed. In Table 10 below the data are shown, summarising the area ratio of D-pinitol/internal standard per 100 ml sample after various extraction cycles on an ultrasonic vibrating bath.

TABLE 10

| | 50 ml-30 min | 100 ml-30 min | 50 ml-1 h | 100 ml-1 h |
|---|---|---|---|---|
| 1 | 0.488 | 0.492 | 0.627 | 0.620 |
| 2 | 0.530 | 0.535 | 0.542 | 0.568 |
| mean | 0.509 | 0.514 | 0.585 | 0.594 |

FIG. 11 is a graphical representation of this, in which 1 represents 50 ml-30 min, 2 represents 50 ml-1 h, 3 represents 100 ml-30 min and 4 represents 100 ml-1 h, respectively.

When a volume of 50 ml is chosen, the ratio does not increase further when the volume is increased. However, there is a significant difference between extracting for 30 minutes or 1 hour, as Table 10 and FIG. 11 show. Nevertheless, the extraction in this way is still not complete, since the ratio is still less than that after four extractions in 10 ml methanol.

It is apparent from the preceding experiments that principally the time versus the number of extractions is an important factor. However, when a sample is in the vibrating bath, this also heats up, so that the heat factor should also be pursued. Table 11 below gives an overview of the D-pinitol/internal standard area ratio for a 100 mg sample after various extraction cycles in a heated ultrasonic vibrating bath.

TABLE 11

| | 1x extraction | 2x extraction | 3x extraction | 4x extraction |
|---|---|---|---|---|
| 1 | 0.634 | 0.670 | 0.646 | 0.670 |
| 2 | 0.631 | 0.702 | 0.716 | 0.716 |
| mean | 0.632 | 0.686 | 0.681 | 0.693 |

FIG. 10 is a graphical representation of this, wherein 1 represents 1× extraction, 2 represents 2× extraction, 3 represents 3× extraction and 4 represents 4× extraction, respectively.

On average, extraction in a heated vibrating bath provides a maximum yield after 2× extraction. After 1, 3 or 4 extraction cycles the ratio does not increase further. The ratio after two cycles of heated ultrasonic vibration is considerably greater than after 1×1 hour of extraction without heat, as would have been expected based on Table 11 and FIG. 11. To pursue the effect of heat, another technique, refluxing, was also used. The table below gives an overview of the area ratio of D-pinitol/internal standard per 100 mg sample after various extraction cycles on a heated ultrasonic vibrating bath.

TABLE 12

| | 1x | 2x | 3x | 4x | |
|---|---|---|---|---|---|
| | refluxing | refluxing | refluxing | refluxing | 1x 1 h refluxing |
| 1 | 0.708 | 0.711 | 0.674 | 0.718 | 0.722 |
| 1b | 0.717 | — | — | — | — |
| 2 | 0.709 | 0.698 | 0.722 | 0.660 | 0.710 |
| mean | 0.711 | 0.705 | 0.698 | 0.689 | 0.716 |

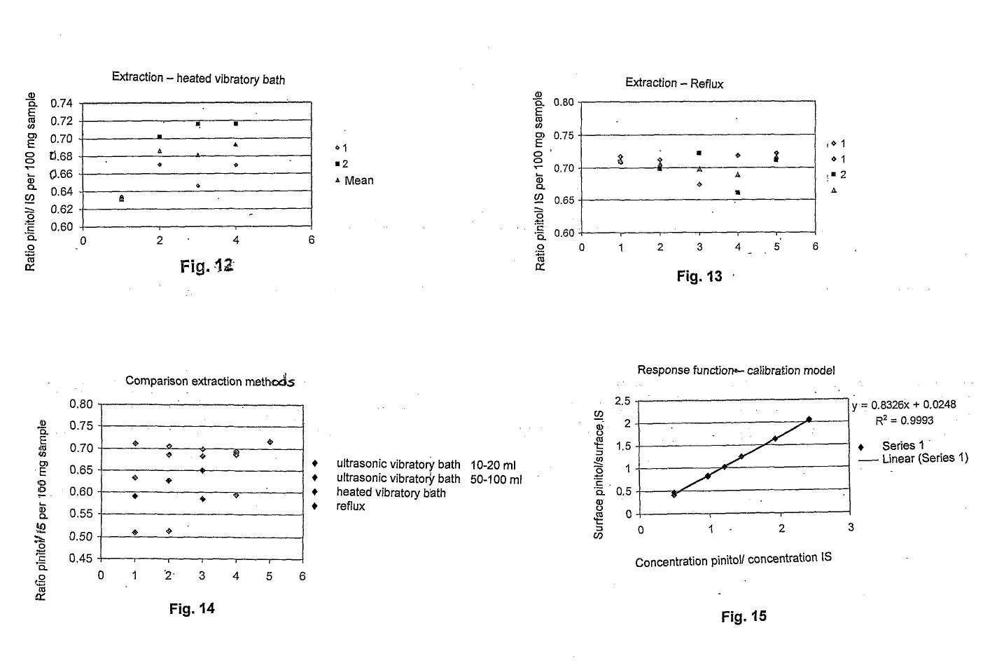

The ratios obtained by refluxing are similar after 1, 2, 3 and 4 cycles of refluxing and also when refluxing is performed once for 1 hour as shown in Table 12 and FIG. 13. To approach the range of these values it would be necessary to perform vibration at least twice in an ultrasonic vibrating bath. The values obtained by refluxing are also just a bit higher. Whether this is coincidence cannot be determined from these two experiments. Nevertheless, it is apparent that refluxing is the most productive and least labour-intensive extraction method, as shown in FIG. 14.

In the development of a final method, initially a look was taken at the internal standard solution, wherein 10.5 mg xylitol was weighed into a volumetric flask on a balance and dissolved in methanol. It was diluted to 100 ml. 25 ml of this solution were pipetted into a 250 ml volumetric flask and diluted to 250 ml. The quantity of internal standard gave a D-pinitol/xylitol ratio in the sample=1.

Then for sample preparation 100 mg of the decoction was weighed into a round-bottom flask. Then 50 ml of the internal standard solution was added. This mixture was refluxed for 30 min. After cooling this was transferred to a tube and centrifuged for 5 minutes at 3000 g. The supernatant was transferred to a receiving vessel. Then 150 μl of this was pipetted into a reaction vial and evaporated to dryness under a stream of nitrogen. Then 50 μl of derivatisation reagent (BSTFA 1% TMCS—pyridine 2:1) were added and vortexed. The reaction vials were heated for 1 h in the heating block at 70° C. After cooling the vials, the contents were transferred to vials that are compatible with the GC auto-injector.

The following temperature gradient was used for the analysis: the temperature was 65° C. for the first two minutes, then the temperature was raised to 300° C. at the rate of 6° C./min. Then 300° C. was maintained for 15 minutes. A gas with a flow rate of 1.3 ml/min was used.

Now the validation of the method. The validation of analytical methods is performed according to the ICH guidelines. According to these guidelines, the linearity, reproducibility and intermediate precision, accuracy, specificity and range of an assay are to be evaluated.

In determination of the calibration model and range, linearity is defined in that results are obtained which are equivalent to the concentration of the analyte in the sample. The range is the interval between the lowest and the highest concentration of analytes within which it is shown that the analytical method is accurate, precise and linear.

The response function was determined by injecting 5 standards at concentrations between 40 and 200% of the theoretical value in duplicate. To determine whether the calibration model is linear, the calibration curve is inspected visually and a linear regression analysis is performed. FIG. 15 shows that the response curve appears linear and thus is a straight line.

TABLE 13
t-test on the slope

| | |
|---|---|
| Theoretical t-value | 2,2281392 |
| Calculated t-value | 119,3243 |

95% CI intersection point (0.0

| Lowest 95% | Highest 95% |
|---|---|
| 0.000770923 | 0.048859935 |

Table 13 illustrates the application of regression analysis with a t-test on the slope and 95% confidence interval intersection point (0,0). It is apparent from the regression analysis that the correlation coefficient, 0.999298, is high enough, in other words >0.99. It is apparent from Table 13 that there is a significant slope from the right, which is also apparent visually in FIG. 15. When the 95% confidence interval is calculated for the intersection point it is clear that the straight line does not pass through (0,0).

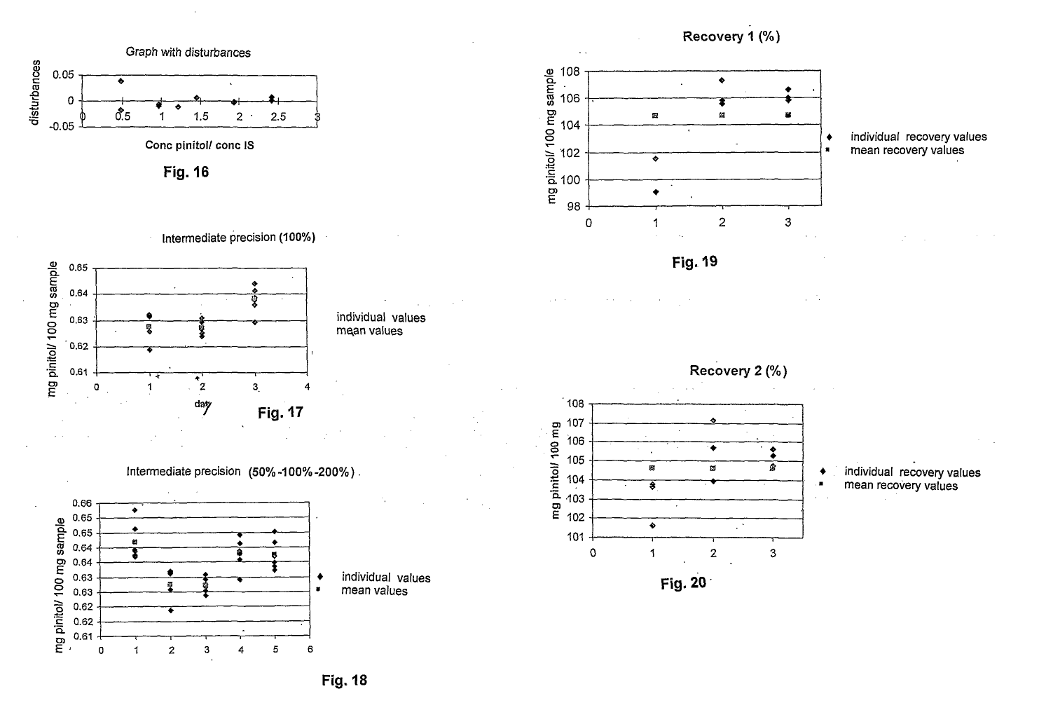

The residuals yᵢ-<yᵢ> are plotted against xᵢ or <yᵢ> to determine whether the residuals are randomly distributed, in other words, that homoscedasticity applies. FIG. 16 shows that the residues are uniformly distributed and the model is homoscedastic. The residue with the greatest deviations still has a deviation of less than 5% relative to the expected value.

An ANOVA lack of fit test was performed to determine whether the model is correct. When the average of the two measured values—for each concentration—deviates too much from the calibration curve with respect to the variance between the two measured values, the F-value would be greater than the critical one, and the model is erroneously selected. The calculated F value is 0.7 and therefore smaller than 4.534. All these data show that the calibration model is linear.

With regard to the precision or repeatability, when analyses are performed with a given method on the same sample, it is expected that the method always gives the same results. For this, the precision or repeatability of the method is verified on different levels. The repeatability of the injection was determined by injecting the same sample 6 times. On three different days, 6 samples were analyzed so as to check repeatability within a day and the intermediate precision.

Also at different concentration levels, namely the lowest and the highest concentration in the range, e.g. 50% & 200% of the theoretical value, the precision was analyzed.

In order to analyze the repeatability of the injection, the following parameters were calculated from the measurement results: the average, the standard deviation and the relative standard deviation.

Table 14 below shows the D-pinitol rates for six injections of the same sample with mean, standard deviation and relative standard deviation expressed in %. It follows from this table that the standard deviation and relative standard deviation are very small, i.e. that the injection is repeatable.

TABLE 14 [symbol]
te D-pinitol (%)

| | |
|---|---|
| 1 | 0.6329 |
| 2 | 0.6308 |
| 3 | 0.6279 |
| 4 | 0.6297 |
| 5 | 0.6328 |
| 6 | 0.6302 |
| Mean | 0.6307 |
| s | 0.0019 |
| RSD | 0.3025 |

[symbol]

indicates data missing or illegible when filed

TABLE 15 [symbol]
te pinitol (%)

| | day 1 | day 2 | day 3 |
|---|---|---|---|
| 1 | 0.6316 | 0.6252 | 0.6360 |
| 2 | 0.6187 | 0.6307 | 0.6292 |
| 3 | 0.6313 | 0.6292 | 0.6412 |
| 4 | 0.6257 | 0.6267 | 0.6378 |
| 5 | 0.6319 | 0.6274 | 0.6386 |
| 6 | 0.6257 | 0.6237 | 0.6441 |
| Mean | 0.6275 | 0.6271 | 0.6378 |
| s | 0.0052 | 0.0026 | 0.0051 |
| RSD % | 0.8264 | 0.4103 | 0.7970 |
| total Mean | 0.6308 | | |
| s total | 0.0066 | | |
| RSD % total | 1.0442 | | |

[symbol]

indicates data missing or illegible when filed

In terms of reproducibility and intermediate precision, in addition to the same calculations as for the precision of the injection, the 95% confidence interval, the intra-day and inter-day variation are calculated. For this purpose, a unifactorial analysis of variance was performed, i.e., an ANOVA single factor test. For this purpose, it was necessary first to check whether the variances of the groups do not differ significantly from one another, otherwise the ANOVA test may not be used.

Table 15 below gives an overview of the D-pinitol content on the various days with mean, standard deviation and relative standard deviation expressed in %.

Looking at this table, it appears that the standard deviations are small and that for day two is smaller, approximately by half, than that of the other days. The coefficients of variance are also relatively small. To determine whether there is a difference between the results for the different days, an ANOVA test was performed. Prior to performing this test it is necessary to determine whether the variances are equal. The Cochran test is used for this, for which the formula (1) is given below:

$$C = \frac{{S}_{max}^{2}}{{Σ}_{j} {S}^{2}} \qquad ( 1 )$$

Table 16 below shows a summary of the variances for the various days and calculated and critical Cochran values.

TABLE 16

| | Variance |
|---|---|
| Day 1 | 0.00002689 |
| Day 2 | 0.00000662 |
| Day 3 | 0.00002584 |
| Critical Cochran value | 0.707 |
| Calculated Cochran value | 0.4530 |

This table shows that the calculated Cochran value is below the critical value. In other words, the variances are considered equal, which means that an ANOVA test can be performed.

Table 17 below gives an overview of the analysis of variance: sum of the squares, degrees of freedom, mean squares and F-values.

TABLE 17

| | Sum of | | |
|---|---|---|---|
| Source of variation | squares | Degrees of freedom | Mean squares |
| Between groups | 0.000440842 | 2 | 0.000220421 |
| Within groups | 0.000296754 | 15 | 1.97836E−05 |
| Total | 0.000737595 | 17 | |

| F | P-value | Critical range of F-test |
|---|---|---|
| 11.14161558 | 0.001082263 | 3.682316674 |

The calculated F-value is higher than the critical F-value. From this, it can be concluded that there is a difference between the results on the different days. To determine whether the deviation is still acceptable, the RSD %_{between} is compared with the ⅔ RSD %_{Horwitz}, which gives an estimate of the maximum RSD % that can be made within a single laboratory. Formula (2) thus considers only the concentration and is as follows:

RSD %_{Horwitz}=2^{(1-0.5 log C)} where C is the concentration (m/m) (2)

Table 18 below shows standard deviations, relative standard deviations expressed in percent, RSD_{Horwitz} and maximum relative standard deviations.

TABLE 18

| | |
|---|---|
| s within | 0.00445 |
| RSD within | 0.70510 |
| s between | 0.01367 |
| RSD between | 2.16770 |
| RSD Horwitz | 4.28724 |
| RSD max | 2.85816 |

This table shows that the RSD % between is smaller than the RSDmax, from which it can be concluded that the method is still precise despite the fact that differences are seen versus the ANOVA test. Since the RSD % on the results within a single day is very small, the chance is increased that a significant difference will be found with the ANOVA test. FIG. 17 is a graphical representation of the individual measurements and mean values on various days, showing that the measurements overlap.

TABLE 20

| | Variance |
|---|---|
| 50% | 0.00003907 |
| 100% | 0.00002689 |
| 100% | 0.00000662 |
| 100% | 0.00002584 |
| 200% | 0.00002514 |
| Critical Cochran value | 0.5060 |
| Calculated Cochran value | 0.3162 |

TABLE 19
Rate pinitol (%)

| | 50% | 100% | 100% | 100% | 200% |
|---|---|---|---|---|---|
| 1 | 0.6374 | 0.6316 | 0.6252 | 0.6360 | 0.6322 |
| 2 | 0.6386 | 0.6187 | 0.6307 | 0.6292 | 0.6416 |
| 3 | 0.6391 | 0.6313 | 0.6292 | 0.6412 | 0.6370 |
| 4 | 0.6462 | 0.6257 | 0.6267 | 0.6378 | 0.6349 |
| 5 | 0.6370 | 0.6319 | 0.6274 | 0.6386 | 0.6452 |
| 6 | 0.6526 | 0.6257 | 0.6237 | 0.6441 | 0.6337 |
| Mean | 0.6418 | 0.6275 | 0.6271 | 0.6378 | 0.6374 |
| s | 0.0063 | 0.0052 | 0.0026 | 0.0051 | 0.0050 |
| RSD % | 0.9739 | 0.8264 | 0.4103 | 0.7970 | 0.7867 |
| total mean | 0.6343 | | | | |
| s total | 0.0076 | | | | |
| RSD % total | 1.1981 | | | | |

The intermediate precision at various concentration levels will be examined in the following. In Table 19 above the contents of D-pinitol in 50 mg, 100 mg and 200 mg samples are shown, with L5 the mean, standard deviation and relative standard deviation for each series. Thus this table 19 gives an overview of the contents of D-pinitol on the various days with mean, standard deviation and relative standard deviation expressed in %.

It is apparent from this table that the errors in the standard deviation for all mean values are in the same order of magnitude everywhere. To determine whether there is a significant difference between the measurements at various concentration levels, here also an ANOVA test was performed after it was confirmed that the variations are not significantly different.

Table 20 above shows a summary of the variances for the different days and calculates a critical Cochran value.

Here also the calculated Cochran value is below the critical value, from which it can be concluded that no significant difference can be demonstrated between the variances of the various groups.

Table 21 below shows an overview of the analysis of variance: sum of squares, degrees of freedom, mean squares and F-values.

TABLE 21

| Source of | | | |
|---|---|---|---|
| variation | Sum of squares | Degrees of freedom | Mean squares |
| Between groups | 0.001057149 | | 0.000264287 |
| Within groups | 0.000617836 | | 2.47134E−05 |
| Total | 0.001674985 | | |

| F | P-value | Critical range of F-test |
|---|---|---|
| 10.69407953 | 3.42484E−05 | 2.758710593 |

It follows from the ANOVA test in table 21 above that the calculated F-value is greater than the critical F-value and that there is thus a significant difference between the different groups. Graphically, the results at 50% and 200% do not overlap with all results at 100%. Nevertheless, the variation between the groups is still acceptable for the method, since the relative standard deviation expressed in percent, the coefficient of variance, is less than the RSDₘₐₓ, the maximum deviation that may be found in a lab.

Table 22 below shows the standard deviations, relative standard deviations expressed in percent, RSD_{Horwitz} and maximum relative standard deviations.

TABLE 22

| | |
|---|---|
| s within | 0.0050 |
| RSD within | 0.7837 |
| s between | 0.0080 |
| RSD between | 1.2675 |
| RSD Horwitz | 2.1418 |
| RSD max | 1.4279 |

Looking at the graph of FIG. 16, it can be concluded that there is no trend in terms of the concentration levels. It can also be concluded from this that sufficient solvent is used and there is no problem with the solubility, otherwise the values would increase as the number of mg of the sample decreased.

As far as the accuracy is concerned, three types of test setups are possible to find out whether the value measured is also the correct value: the test mixture method, the method of standard additions and comparison with a generally accepted method. Only the method of standard additions can be applied here, since no reconstituted product can be made from the plant material, since not all constituents are known, and there is not yet an acceptable method available, since one needs to be developed. The method of standard additions means that a quantity of sample is added to a known quantity of standard solution. Then a determination is made of how much of the substance is recovered using the following formula:

$$Recovery ( \% ) = \frac{{X}_{after} - {X}_{before}}{{X}_{added}} \times 100$$

Table 23 below shows the recovery values with mean, standard deviation, relative standard deviation and 95% confidence interval.

TABLE 23

| | added (%) | recovery (%) |
|---|---|---|
| 1 | 50 | 99.05483058 |
| 1 | 50 | 101.5438868 |
| 2 | 100 | 107.2875403 |
| 2 | 100 | 105.8382079 |
| 2 | 100 | 105.5938629 |
| 3 | 125 | 106.5970772 |
| 3 | 125 | 105.8311879 |
| 3 | 125 | 106.0000916 |
| Mean | | 104.72 |
| s | | 2.858 |
| RSD | | 2.729 |
| CI | | [102.34-107.12] |

TABLE 24

| | added (%) | recovery (%) |
|---|---|---|
| 1 | 50 | 101.61 |
| 1 | 50 | 103.65 |
| 1 | 50 | 103.77 |
| 2 | 100 | 103.95 |
| 2 | 100 | 105.69 |
| 2 | 100 | 107.18 |
| 3 | 125 | 104.73 |
| 3 | 125 | 105.26 |
| 3 | 125 | 105.64 |
| Mean | | 104.61 |
| s | | 1.594 |
| RSD | | 1.524 |
| CI | | [103.38-105.84] |

The corresponding FIG. 19, a graphical representation of the values recovered according to the method of standard additions, wherein 1=50% added, 2=100% added, 3=125% added.

Table 24 below shows the recovery values with mean, standard deviation, residual standard deviation and 95% confidence interval.

As is likewise apparent from the corresponding FIG. 20, which illustrates the values found using the method of standard additions, wherein 1=50% added, 2=100% added, 3=125% added, the values in which 50% were added are somewhat lower than when 100% or 125% was added. To determine whether this is simply variability, the experiment must be repeated again. In general, somewhat more than 100% was recovered, which is to be expected when analyses are conducted. It should also be noted that the recovery experiment only shows relative systematic errors and not absolute ones, since the matrix is the same before and after addition.

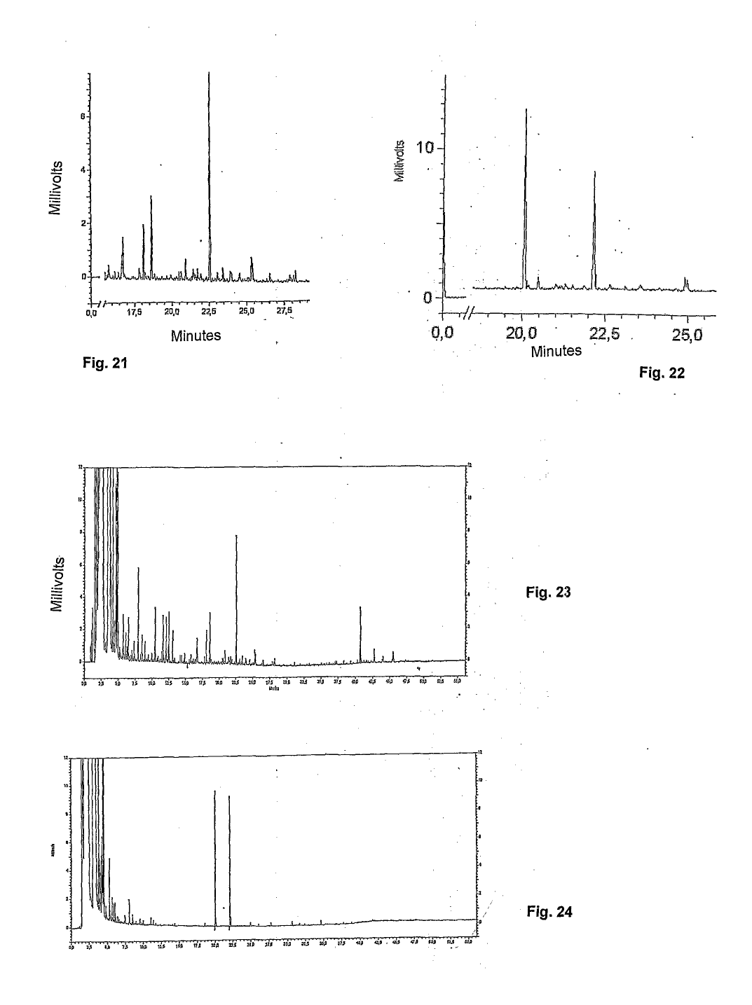

To determine the specificity, the analysis was repeated with mass spectrometric detection to determine whether only the analyte was measured and no other substances. The analysis was also performed without an internal standard to determine whether no other substances were eluted at the same time. FIG. 21 shows a partial chromatogram of the analysis without internal standard. No interferences were visible at 20 to 21 minutes.

FIG. 22 shows a partial chromatogram of the analysis with internal standard. No interferences were present at the location of xylitol in the chromatogram.

The mass spectroscopic analysis also confirmed that the method is specific. The corresponding mass spectra are shown in the respective FIG. 23 ff, wherein FIG. 23 is a complete chromatogram of the analysis without internal standard, showing the voltage in mV versus minutes.

FIG. 24 shows a complete chromatogram of the analysis of the standards with voltage in mV versus minutes, where the peak at Rₜ=20 min is xylitol while the peak at Rₜ=22.45 is D-pinitol.

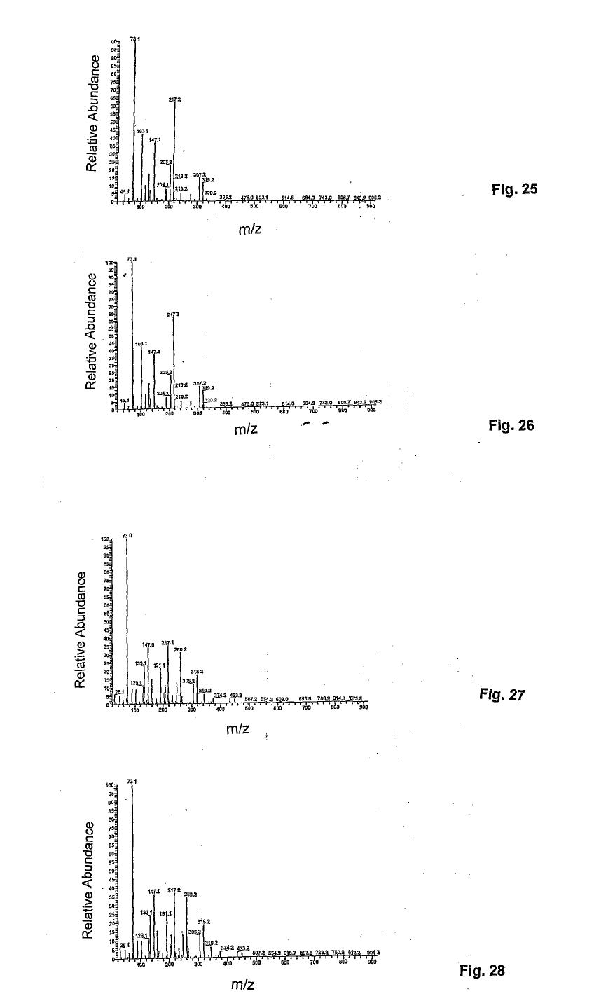

FIGS. 25 to 28 show respectively the specificity of a mass spectrum of standard xylitol; xylitol in the sample; D-pinitol sample and standard D-pinitol, showing the relative abundance in percent as a function of the highest signal.

As far as the in vivo evaluation of the hepato-protective effect of Desmodium adscendens decoction is concerned, D-pinitol has a hepato-protective effect. One of the constituents of Desmodium adscendens is D-pinitol. For the in vivo evaluation of the hepato-protective effect of Desmodium adscendens decoction it appears from previous analyses that the decoction of the plant originally contains about 0.65% D-pinitol, although this is no longer representative. In this in vivo experiment the preventive effect of a decoction of Desmodium adscendens against liver injury induced by galactosamine was investigated in rats. Silymarin was used as the reference agent. Silymarin is a mixture of various flavanolignans from the fruits of the milk thistle plant, Silybum marianum. The principal components are silybin, silychristine and silydianine. In addition, small quantities of isosilybin are present in milk thistle.

Thus a number of in vivo experiments were performed in test animals with the goal of optimising the dose administered and the treatment schedule and looking for a possible effect on liver injury by ethanol and acetaminophen.

In the experiment performed, the following scheme was used for six groups of test animals:

Desmodium decoct 20 mg/kg;

Desmodium decoct 5 mg/kg;

D-pinitol 20 mg/kg;

Silymarin 20 mg/kg (positive control);

No treatment, galactosamine intoxication with single dose 650 mg/kg (negative control); No treatment, no intoxication.

The treatment schedule was as follows:
Day 0: for treatment with Desmodium decoct: 20 mg/kg or 5 mg/kg
   pretreatment with D-pinitol: 20 mg/kg
   pre-treatment with silymarin: 20 mg/kg
Day 1: pre-treatment with Desmodium decoct: 20 mg/kg or 5 mg/kg
   pretreatment with D-pinitol: 20 mg/kg
   pre-treatment with silymarin: 20 mg/kg
   immediately followed by administration gelactosamine
Day 2: all blood groups (24 h)
   treatment with Desmodium decoct: 20 mg/kg or 5 mg/kg
   Treatment with D-pinitol: 20 mg/kg
   treatment with silymarin: 20 mg/kg
Day 3: all blood groups (48 h).

The following 3 control groups had to be present in each experiment:
Silymarin 20 mg/kg (positive control);
no treatment, only galactosamine intoxication 650 mg/kg (negative control);
no treatment, no intoxication.

In the experiment performed, the effect after 24 h did not yet seem very pronounced, but it was after 48 h. Therefore, no blood samples were taken at 24 h in the follow-up experiments, but only after 48 h, followed by a second sample after at least an additional 24 h, and possibly a third blood sample at an even later time.

Optimisation of the dose and treatment schedule was performed in experiments with D-pinitol. Then this dose was calculated based on Desmodium decoction for another experiment.

To determine the hepato-protective effect of the preparation being tested, therefore, the selected experimental animals, namely Sprague Dawley rats, underwent pre-treatment on day 0 and day 1 before the hepatotoxic agent was administered (day 1). On day 2 there was also a single after-treatment. The Desmodium decoction, the pure active ingredient D-pinitol, and the positive control silymarin were all administered orally by gavage.

This resulted in the aforementioned 6 test groups, namely:

Control No hepatotoxic agent (vehicle), no treatment (vehicle)

Hepatotox: Hepatotoxic effect, no treatment (vehicle)

Desmodium 1: Hepatotoxic effect, treatment with Desmodium equivalent to 20 mg/kg D-pinitol

Desmodium 2: Hepatotoxic effect, treatment with Desmodium equivalent to 5 mg/kg D-pinitol

D-pinitol: Hepatotoxic effect, treatment with pure D-pinitol 20 mg/kg

Silymarin: Hepatotoxic effect, treatment with silymarin 20 mg/kg.

In summary, the treatment schedule was as follows: on day 0 and day 1 a pre-treatment was given with one of the test preparations. After the pre-treatment, the groups of experimental animals in question were given a hepatotoxic product on day 1: D-galactosamine 650 mg/kg IP 2% in physiol. solution. On day 2 a treatment dose was likewise administered. On day 2 and day 3, 1.5 ml blood was collected from the tail vein. The liver injury and the possible hepato-protective effect were evaluated by determining the following parameters in serum: ALT (=GPT), AST (=GOT), ALP. The results of the tests were as follows; see the respective tables and FIG. 29 ff. After administration of the hepatotoxic agent D-galactosamine, both after 24 h and after 48 h, liver injury was observed through a marked increase in the three parameters determined (control vs. hepatotox).

After 24 h, no significant decrease in AST (GOT) and ALP was seen for any treatment compared with the hepatotoxic group, thus also not for the positive control silymarin. A significant decrease was seen in ALT (GPT) in both Desmodium groups and with silymarin.

After 48 hours, the results were more pronounced: both AST (GOT) and ALT (GPT) had decreased significantly compared with the hepatotoxic group for all treatments: both Desmodium dosages, D-pinitol and silymarin. A few noteworthy observations were that the lowest Desmodium dosage (equivalent to 5 mg/kg D-pinitol) already has a very pronounced effect, which is comparable to the highest dose (equivalent to 20 mg/kg D-pinitol); that both Desmodium doses are more active than, or at least as active as, silymarin; and that both Desmodium doses are more active than or at least as active as 20 mg/kg pure D-pinitol.

As far as the parameter ALP is concerned, after the treatment was administered, no effect could yet be seen. Perhaps longer treatment is required for this.

Thus it can be concluded that the hepato-protective effect of the standardised Desmodium adscendens preparation is clearly demonstrated in the treatment schedule used. Further study on the dose-dependence of the effect is indicated, in view of the good results obtained with the lowest dose of Desmodium decoction. The effect of longer pre-treatment, of a therapeutic treatment that only starts after administration of the hepatotoxic agent, and a combination of the two can be further investigated; possibly on a broader set of liver injury parameters.

Another experiment consisted of the design and execution of an experiment with ethanol-induced liver injury instead of galactosamine, with the same doses of Desmodium decoction as in the previous experiment.

The anti-hepatotoxic activity of a standardised Desmodium adscendens decoction and D-pinitol against chemically induced liver injury in rats was investigated, in particular the protective effect of D. adscendens decoction against ethanol-induced liver injury.

The purpose of the experiment is to evaluate the hepatoprotective effects of decoct of Desmodium adscendens against ethanol-induced liver injury, standardized to its primary ingredient: D-pinitol. Materials and Methods: 66 male Wistar rats of 200-225 g (Charles River, Brussels, Belgium) were randomly divided into 6 groups: the negative control group (CON: no hepatotoxic agent, no treatment: 8 rats), the hepatotoxic group (HEP: induction of hepatotoxicity, no treatment: 20 rats), the Desmodium I group (induction of hepatotoxicity, treatment with Desmodium adscendens decoct, equivalent to 2 mg/kg of D-pinitol: 10 rats), the group II Desmodium (induction of hepatotoxicity, treatment with Desmodium adscendens decoct, equivalent to 10 mg/kg of D-pinitol: 10 rats), the D-pinitol group (PIN: induction of hepatotoxicity, treatment with 10 mg/kg of D-pinitol: 10 rats), the positive control group (SIL: induction of hepatotoxicity, treatment with 20 mg/kg of silymarin: 8 rats). 2 days prior to ethanol administration, an appropriate amount of lyophilized decoct, pinitol silymarin or suspended in water and administered by gavage (or vehicle for the control group). Hepatotoxicity was induced by daily oral gavage with a 55% ethanol solution (except for the negative control group). An initial dose of 2 g/kg of ethanol was gradually increased to a dose of 6 g/kg during the first week of the experiment. Ethanol was administered for a period of 7 weeks. Was administered daily, depending on the group, treated with the specific decoct, pinitol or silymarin (oral gavage) to the end of the experiment. For ethanol administration (default values), and at weeks 3, 4, 5 and 6, blood samples (1.5 ml) were taken from the lateral tail vein (Multi Fat 600, Sarstedt). This experiment was approved by the Ethics Committee for Animal Experiments of the University of Antwerpen (17-01-2011, 2010-37).

CON: no ethanol, no treatment
HEP: ethanol, no treatment
DES 1: ethanol, Desmodium adscendens treatment equivalent to 2 mg/kg
of D-pinitol
DES 2: ethanol, Desmodium adscendens treatment equivalent to 10 mg/kg
of D-pinitol
PIN: ethanol, D-pinitol treatment (10 mg/kg)
SIL: ethanol, silymarin treatment (20 mg/kg)

A schematic overview of the different treatment groups is given below next. Concentrations of enzyme aspartate transaminase (AST, GOT) and alanine transaminase (ALT, GPT) were in the serum samples from the test animals as determined by routine laboratory techniques (Senior, 2009). Elevated levels can be considered as an indication of liver cell destruction or a change in membrane permeability.

Statistics to analyze the difference between CON and HEP for AST and ALT, and between HEP and DES 1, DES 2, PIN and SIL for AST and ALT, was a mixed model analyze performed. The log-rank test was performed for statistical analysis of survival data.

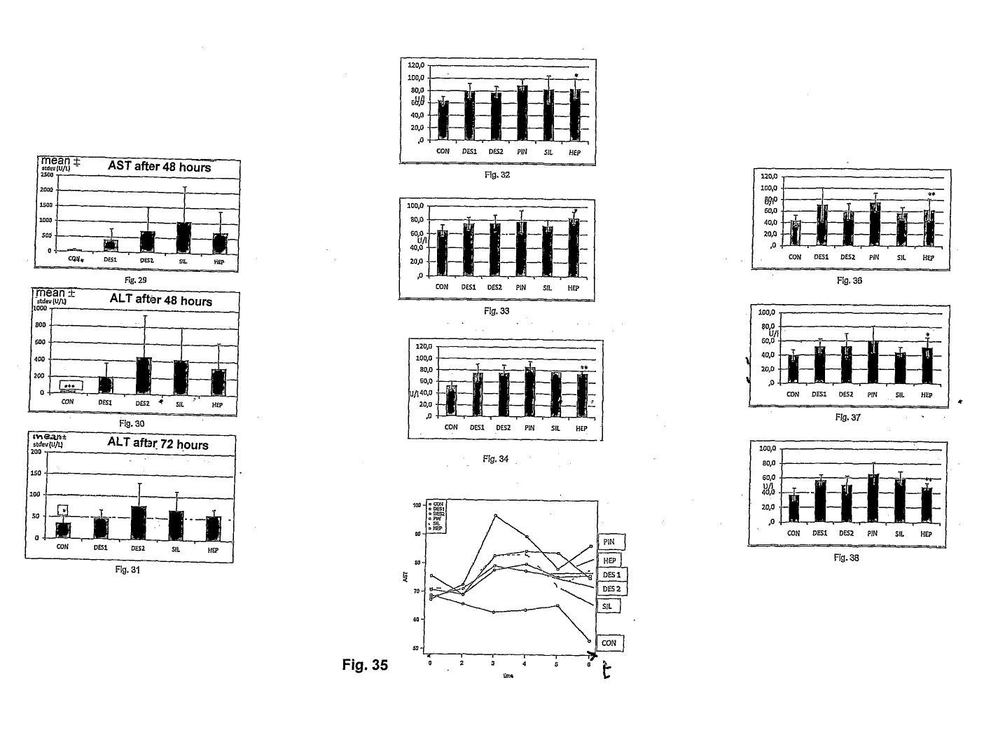

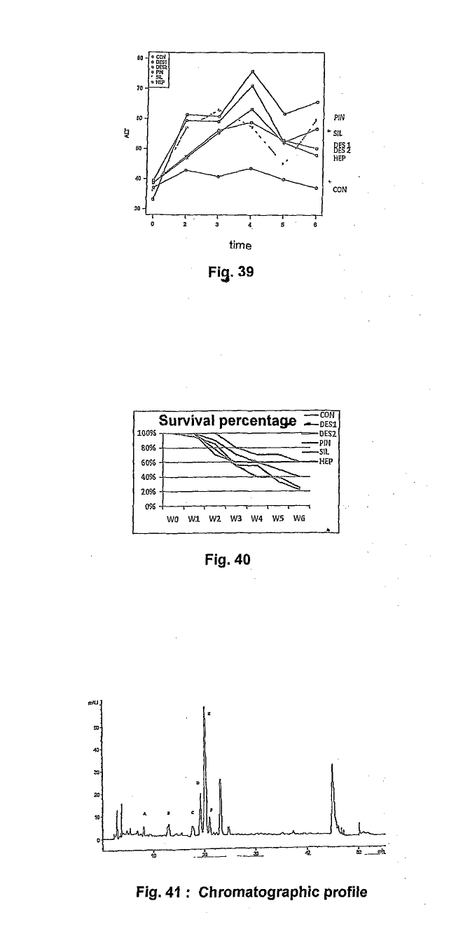

Results: Serum AST and ALT values in weeks 4, 5 and 6 of the study are shown in Tables 26-31, and FIG. 32-34, 36-38. Average AST and ALT values at different points in time (weeks 0, 2-6) are shown in FIGS. 35 and 39. Tables 32 and 33 and FIG. 40 show the survival time in the different animal groups.

TABLE 27
AST_5

| | Average | | SEM |
|---|---|---|---|
| | (U/I) | (U/I) | (U/I) |
| CON | 65.3 | 7.4 | 2.5 |
| DES1 | 75.4 | 8.6 | 3.5 |
| DES2 | 75.5 | 11.5 | 4.7 |
| PIN | 78.3 | 15.8 | 7.1 |
| SIL | 72.1 | 8.6 | 5.0 |
| HEP | 83.8 | 8.7 | 2.7 |
| | | ↓ | |

Standard-deviation

TABLE 26
AST_4

| | Average | | SEM |
|---|---|---|---|
| | (U/I) | (U/I) | (U/I) |
| CON | 63.6 | 6.7 | 2.4 |
| DES1 | 79.8 | 12.3 | 5.5 |
| DES2 | 77.3 | 9.9 | 3.7 |
| PIN | 89.3 | 9.3 | 3.8 |
| SIL | 83.2 | 21.7 | 9.7 |
| HEP | 84.2 | 16.3 | 5.1 |
| | | ↓ | |

Standard-deviation

Table 26 above right shows Serum AST values after 4 weeks of ethanol administration. FIG. 32 shows Serum AST values after 4 weeks of ethanol administration, * p<0.05, ** p<0.01, *** p<0.001 vs HEP CON; † p<0.05 vs HEP DES 1, DES2, PIN, SIL (Mixed model analysis).

Table 27 above left shows Serum AST values after 5 weeks of ethanol administration.

FIG. 33 shows Serum AST values after 5 weeks of ethanol administration, * p<0.05, ** p<0.01, *** p<0.001 vs HEP CON; † p<0.05 vs HEP DES 1, DES2, PIN, SIL (Mixed model analysis).

Table 28 below right shows serum AST levels after 6 weeks of ethanol administration.

| | | |
|---|---|---|
| Serum | ALT | ALT_4 |
| Average | Standard Deviation | SEM |

| | | | |
|---|---|---|---|
| CON | 43.6 | 9.1 | 3.0 |
| DES1 | 71.0 | 29.1 | 11.9 |
| DES2 | 58.8 | 14.0 | 5.3 |
| PIN | 75.9 | 15.5 | 6.3 |
| SIL | 57.4 | 9.9 | 4.4 |
| HEP | 63.2 | 19.0 | 5.7 |

AST_6

| | Mean | Standard deviation | SEM |
|---|---|---|---|
| | (U/I) | (U/I) | (U/I) |
| CON | 53.1 | 6.8 | 3.0 |
| DES1 | 75.9 | 15.3 | 6.3 |
| DES2 | 75.9 | 13.2 | 5.4 |
| PIN | 86.3 | 9.4 | 4.7 |
| SIL | 77.8 | .3 | .2 |
| HEP | 75.0 | 4.1 | 2.0 |

FIG. 34 shows serum AST levels after 6 weeks of ethanol administration, * p<0.05, ** p<0.01, *** p<0.001 vs HEP CON; † p<0.05 vs HEP DES 1, DES2, PIN, SIL (Mixed model analysis).

FIG. 35 shows AST Average values per time point (weeks 0, 2, 3, 4, 5, 6).

Table 29 above shows Serum ALT levels after 4 weeks of ethanol administration.

ALT_6

| | Mean | Standard.Deviation | Standard.Error.of.Mean |
|---|---|---|---|
| CON | 37.0 | 9.1 | 4.1 |
| DES1 | 56.6 | 7.7 | 3.1 |
| DES2 | 50.1 | 12.2 | 5.0 |
| PIN | 65.6 | 15.7 | 7.8 |
| SIL | 59.7 | 9.3 | 6.6 |
| HEP | 47.9 | 5.3 | 2.6 |

ALT_5

| | Mean | Standard.Deviation | Standard.Error.of.Mean |
|---|---|---|---|
| CON | 39.8 | 7.6 | 2.5 |
| DES1 | 52.3 | 10.7 | 4.4 |
| DES2 | 52.8 | 17.5 | 6.6 |
| PIN | 61.6 | 19.2 | 8.6 |
| SIL | 44.8 | 6.9 | 4.0 |
| HEP | 52.0 | 13.4 | 4.2 |

Table 30 above shows Serum ALT levels after 5 weeks of ethanol administration.

FIG. 36 shows serum ALT values after 4 weeks of ethanol administration, * p<0.05, ** p<0.01, p<0.001 vs HEP CON; † p<0.05 vs HEP DES 1, DES2, PIN, SIL (Mixed model analysis).

FIG. 37 shows serum ALT levels after 5 weeks of ethanol administration, * p<0.05, ** p<0.01, *** p<0.001 vs HEP CON; † p<0.05 vs HEP DES 1, DES2, PIN, SIL (Mixed model analysis).

Table 31 above shows serum ALT levels after 6 weeks of ethanol administration.

Fig. * P<0.05; ** p<0.01, FIG. 38 shows serum ALT levels after 6 weeks of ethanol administration, *** p<0.001 vs HEP CON; † p<0.05 vs HEP DES 1, DES2, PIN, SIL (Mixed model analysis).

FIG. 39 shows ALT Average values per time point (weeks 0, 2, 3, 4, 5, 6).

Mortality

Table 32 shows the survival of test animals in the test groups over a period of 6 weeks.

| | CON | DES1 | DES2 | PIN | SIL | HEP |
|---|---|---|---|---|---|---|
| W0 | 100% | 100% | 100% | 100% | 100% | 100% |
| W1 | 100% | 100% | 100% | 100% | 100% | 95% |
| W2 | 100% | 90% | 100% | 70% | 78% | 85% |
| W3 | 100% | 70% | 80% | 60% | 56% | 55% |
| W4 | 100% | 60% | 70% | 60% | 56% | 40% |
| W5 | 100% | 60% | 70% | 50% | 33% | 40% |
| W6 | 100% | 60% | 60% | 40% | 22% | 25% |

FIG. 40 shows the survival of test animals in the test groups over a period of 6 weeks. Table 33 shows log-rank test for the determination of difference in the survival of the animals over a period of 6 weeks.

TABLE 33

| Comparison | P-value | |
|---|---|---|
| CON-HEP | 0.0044 | [symbol] |
| HEP-DES1 | 0.11 | |
| HEP-DES2 | 0.06 | [symbol] |
| HEP-PIN | 0.56 | |
| HEP-SIL | 4.88 | |
| All treated groups | 0.141 | |

In this experiment, wherein the preventive effect of Desmodium adscendens decoct against ethanol-induced liver injury was evaluated, the hepatotoxic group (HEP) shows significantly increased serum levels of AST and ALT after 4 weeks of daily ethanol administration (FIG. 32-34, 36-38). Treatment with Desmodium adscendens (2 mg/kg and 10 mg/kg), pinitol (10 mg/kg) or silymarin (20 mg/kg) was started 2 days before administration of ethanol and further put daily until the end of the experiment, 6 weeks later. As shown in FIGS. 32-34 and 36-38, no significant reduction was observed with regard to serum AST (FIGS. 32, 33 & 34 after treatment of resp. 4, 5 & 6 weeks), and ALT levels (FIGS. 36, 37 & 38 after treatment of resp. 4, 5 & 6 weeks), for any treatment. Evaluation of the mortality of the animals in the different treatment groups (FIG. 40, Table 32), showed a large drop in the untreated group hepatotoxic (HEP), while survival was better in DES 2. Moreover, after 6 weeks of ethanol-administration, 60% of the rats that were given decoct Desmodium adscendens survived, while the survival rate in the pinitol and silymarin-treated groups was 40%, and respectively 22%. Statistical analysis of the survival data (Table 33) shows a statistically significant difference in survival between control and hepatotoxic group (p<0.01), and a trend towards significance (p=0.06) between the D. adscendens group DES 2 (eq. to 10 mg/kg pinitol) and the untreated group hepatotoxic. No statistically significant difference between the group DES 1 (eq. to 2 mg/kg pinitol), pinitol group (10 mg/kg) group or silymarin (20 mg/kg) and the hepatotoxic group was observed.

Since a period of at least 4 weeks was needed to produce significant ethanol-induced hepatotoxic effects, in rats, and a large drop of the hepatotoxic untreated animals was observed, it was not possible to investigate the hepatocurative effects of Desmodium adscendens decoct in this experiment.

A further experiment consisted in setting up and conducting an experiment with acetaminophen-induced liver damage instead of galactosamine.

The experiment aimed to evaluate the hepato-curative effects of a decoction of Desmodium adscendens, standardized on its main component D-pinitol, against acetaminophen (paracetamol)-induced liver injury.

The materials and methods used for this purpose consisted of 40 male Wistar rats of 200-225 g, which were randomly divided into 5 groups: the negative control group (CON: no hepatotoxic agent, no treatment: 8 rats), and the hepatotoxic group (HEP: induction of hepatotoxicity, no treatment: 8 rats), and the group I Desmodium (induction of hepatotoxicity, treatment with D. adscendens decoct, equivalent to 2 mg/kg of D-pinitol: 8 rats), and the group II Desmodium (induction of hepatotoxicity, treatment with D. adscendens decoct, equivalent to 10 mg/kg of D-pinitol: 8 rats), and the positive control group (SIL: induction of hepatotoxicity, treatment with 20 mg/kg of silymarin: 8 rats). Acute hepatotoxicity was induced by oral gavage with 2 g/kg acetaminophen (except the control group). 24 h after hepatotoxicity induction, treatment was initiated with the appropriate amount (see above), lyophilized extract of D. adscendens or silymarin, suspended in water and administered by gavage (or vehicle for the control groups and hepatotoxic). Blood samples were taken from the lateral take-off vein (1.5 ml, Multi Fat 600, Sarstedt) 24 h, 48 h, and 72 h after acetaminophen administration. This experiment was approved by the Ethics Committee for Animal Experiments of the University Antwerpen (17-01-2011, 2010-37).

A schematic overview of the different treatment groups is shown below:

CON: no acetaminophen, no treatment; HEP: 2 g/kg acetaminophen, no treatment

DES 1: 2 g/kg acetaminophen, D. adscendens treatment equivalent to 2 mg/kg of D-pinitol

DES 2: 2 g/kg acetaminophen, Desmodium adscendens treatment equivalent to 10 mg/kg D-pinitol

SIL: 2 g/kg acetaminophen, silymarin treatment (20 mg/kg).

Enzyme levels of aspartate transaminase (AST, GOT) and alanine transaminase (ALT, GPT) were determined in serum samples from animals with routine laboratory techniques (Senior, 2009).

Increased levels can be regarded as an indication of liver cell destruction or a change in membrane permeability. For statistical purpose, a One way ANOVA was performed, so as to analyze the difference between CON and HEP for AST and ALT, and between HEP and DES 1, DES 2 and SIL for AST and ALT, which was followed by Dunnett post hoc test (SPSS statistics program). The results for serum AST and ALT values at 48 h and 72 h after acetaminophen administration are shown in Tables 34-37, and FIG. 29-31.

TABLE 34

| | AST 48 | | |
|---|---|---|---|
| | | Standaard | |
| | Gemiddel | Deviate | SEM |
| | (U/I) | (U/I) | (U/I) |
| CON | 62 | 9 | 4 |
| DES1 | 403 | 363 | 128 |
| DES2 | 717 | 753 | 266 |
| SIL | 1047 | 1155 | 408 |
| HEP | 704 | 673 | 213 |

TABLE 35

| | AST 72 | | |
|---|---|---|---|
| | | Standaard | |
| | Gemiddel | Deviatie | SEM |
| | (U/I) | (U/I) | (U/I) |
| CON | 62 | 10 | 3 |
| DES1 | 76 | 19 | 7 |
| DES2 | 99 | 38 | 13 |
| SIL | 110 | 47 | 17 |
| HEP | 81 | 12 | 4 |

FIG. 29 shows Serum AST values 48 h after acetaminophen administration, * p<0.05, ** p<0.01, *** p<0.001 vs. CON HEP (Statistics: One-Way ANOVA, post hoc Dunnett versus HEP).

Serum ALT

Table 36 & 37 below show Serum ALT values 48 and 72 h after acetaminophen administration.

| | alt48 | | |
|---|---|---|---|
| | | Standaard | |
| | Gemiddel | Deviatie | |
| | de (U/I) | (U/I) | SEM (U/I) |
| CON | 28 | 6 | 2 |
| DES1 | 208 | 161 | 57 |
| DES2 | 451 | 483 | 171 |
| SIL | 423 | 380 | 134 |
| HEP | 327 | 295 | 93 |

| | alt72 | | |
|---|---|---|---|
| | | Standaard | |
| | Gemiddel | Deviatie | |
| | de (U/I) | (U/I) | SEM (U/I) |
| CON | 36 | 16 | 6 |
| DES1 | 49 | 18 | 6 |
| DES2 | 79 | 52 | 18 |
| SIL | 70 | 42 | 15 |
| HEP | 59 | 13 | 5 |

FIG. 30, 31 show Serum ALT values 48 and 72 h after acetaminophen administration, * p<0.05, ** p<0.01, *** p<0.001 vs. CON HEP (Statistics: One-Way ANOVA, Dunnett post hoc versus HEP). In this experiment, that evaluated the curative effect of Desmodium adscendens decoct against acetaminophen-induced liver damage, the hepatotoxic group (HEP) showed significantly increased levels of serum AST and ALT at 24 h and 48 h after acetaminophen administration (p<0.001), and also at 72 h after acetaminophen administration for ALT (p<0.05). An oral treatment of Desmodium adscendens (2 mg/kg and 10 mg/kg) or silymarin (20 mg/kg, positive control group) was given 24 h and 48 h after acetaminophen administration. As shown in FIG. 29-31 no significant decrease (p>0.05) could be observed for a serum AST and ALT levels in blood sampling points 48 h and 72 h for any treatment. The results of this experiment in a rat model of acute hepatic damage, induced by acetaminophen, show that Desmodium adscendens has no hepatocurative effect when administered in a daily dose of up to 10 mg/kg.

Finally, a so-called Ames test was performed on samples of UA: Desmodium extract. The extract was tested according to the OECD guideline with 5 Salmonella typhimurium strains (TA98, TA100, TA102, TA1535 and TA1537) in the absence and presence of a metabolising S9 fraction. For this experiment, fresh strains were used, also S9 and other needed materials such as NADPH, Petri dishes, etc.

In accordance with the guidelines, the tests were started with a maximum test concentration of 5 mg/plate, after which dilutions were carried out using 6 dosages. A negative (solvent) control was used with 4 Petri dishes, as well as a positive control with 3 Petri dishes. Positive controls are part of the recommended list of controls at the recommended test concentration. A list of the positive controls that we used and their concentrations is available.

Thus three Petri dishes were prepared for each concentration and 4 for the negative control. The results showed the mean values thereof, in particular the mean number of revertants ±SD. The test is successful if the negative and positive concentrations are within the expected range and no signs of toxicity are seen. This was determined by examining the background layer of bacteria, wherein the absence of a background layer indicates toxicity. No signs of toxicity were found, and all strains and controls gave the expected response to TA102. A test substance is positively “mutagenic” if a doubling of the number of revertants is seen compared with the negative (solvent) control, and if a dose-effect relationship is observed.

A synthesis of the results is presented in the following. The above data indicate that the sample is not mutagenic in the strains TA98, TA100 and TA1535, both in the presence and in the absence of S9. For TA1537, just a doubling of the mutation frequency was found in the presence of S9 at the highest concentration, 5 mg/plate, relative to the negative control, but no pronounced dose-effect relationship. To be certain, the test was repeated. No dose-effect relationship was found, and also no doubling with respect to the control, which confirms that there was also no mutagenic effect in strain TA1537.

It could be established experimentally that there is practically no mutagenic effect. The Desmodium extract is not mutagenic in the Ames test either in the absence or in the presence of a metabolising S9 fraction. An increase was seen in the number of spontaneous revertants at the highest dose, but this was nevertheless insufficiently great to use the term “mutagenicity.” Repeated experiments (at least 1 per strain) confirm the absence of mutagenicity.

Regarding flavonoid content and profile in Desmodium adscendens lyophilisate,

In the employed method, there is firstly the sample preparation:

To prepare the standard (reference solution), 500 mg vitexin was weighed into a 10.0 ml volumetric flask and dissolved in methanol. 3 ml of this were pipetted over to a 20.0 ml volumetric flask and this was diluted with 50% methanol.

For preparing the samples (test solutions), in each case about 100 mg powder (lyophilisate) is weighed into a 25.0 ml volumetric flask and 20 ml 50% methanol is added. The solutions are then sonicated for 15 minutes in the ultrasonic bath. After the samples are cooled, they are diluted to 25.0 ml with 50% methanol. Both the test solution(s) and the reference solution(s) are filtered through a nylon syringe filter.

HPLC Conditions

The mobile phases used were A: 1.0% (v/v) phosphoric acid in water and B: acetonitrile. The gradient conditions were as follows:

Start conditions: 5% B

0-5 min.→10% B 5-80 min.→50% B 85-90→5% B

With a flow of 1.0 ml/min. Detection is at 360 nm. Column used is a Grace smart column or Lichrosphere column (250×4.5 microns).

Results: Total content of flavonoids as vitexin in a typical lyophilisate of D. adscendens, wherein all peaks with a UV spectrum typical of flavonoids are totaled: 1.122%

Typical HPLC Profile

The indicated peaks show a UV spectrum characteristic for flavonoids.

Desmodium adscendens Lyophilisate

Expansion to flavonoids with chromatographic profile and content

Method used

Sample preparation see above.

HPLC Conditions'

As mobile phase, A: H2O with 0.1% formic acid and B: acetonitrile are used. The gradient conditions are as follows:

Start conditions: 15% B

0-5 min→15% B 5-39 min→23% B ⋄ ⋄ 39-43 min→100% B

43-45 min→100% B ⋄ 45-47 min→15% B 47-55 min→15% B

with a flow rate of 1.0 ml/min. Detection is at 334 nm (DAD). As a column, a Luna C18 column (Phenomenex) (250×4 mm, 5 um) is used.

Results

Flavonoid profile+identification

With the indicated method, a chromatographic profile was obtained as shown in the Figure. Diode Array Detection (DAD) of the labelled signals (A-F) led to the UV spectra shown. All labelled signals (peak A-peak F) exhibit a UV spectrum that is characteristic of flavonoids.

After isolation of the products involved, the principal component (peak E) was identified as vitexin-2″-xyloside on the basis of mass spectrometry and NMR spectroscopy. The table shows the assignments of the ¹H and ¹³C-NMR spectra compared with published data. In this way it was also possible to identify peak F spectroscopically as vitexin.

Content Determination

The total content of flavonoids, determined as the sum of the labelled signals, expressed as vitexin, in a typical lyophilisate of (the decoction of) D. adscendens was 1.05%.

Table

¹H and ¹³C-NMR assignments for peak E (taken up in DMSO-d₆) and vitexin-2″-xyloside

13C (ppm) 1H (ppm) 13C (ppm) 1H (ppm)

2 163.8 164.2

3 102.5 6.70 102.8 6.80 s

4 181.6 182.4

5 160.6 13.10 (OH) 160.9

6 99.0 6.18 98.5 6.27 s

7 162.5 163.1

8 104.1 104.0

9 156 156.9

10 103.3 104.1
1′ 121.7 121.9

2′, 6′ 128.9 7.99 (d, 8.4 Hz) 8:04 129.2 (d, 8.6 Hz)

3′, 5′ 116.3 6.89 d, 8.4 Hz) 116.3 6.94 (d, 8.6 Hz)

4′ 161.7 161.5

Glc-1 71.9 4.79 (d, 71.9 4.83 (d, 9.8 Hz)

Glc-2 81.7 3.24 82.1 3.32 m

Glc-3 78.8 3.46 78.6 3.58

Glc-4 70.5 3.42 70.5 3.54

Glc-5 80.8 4.06 81.6 4.09 br t

Glc-6 61.2 3.73 61.3 3.79 3:54, 3.60

Xyl-1 106.2 3.88 106.1 3.91 (d, 7.0 Hz)

Xyl-2 73.0 2.75 74.0 2.80 dd

Xyl-3 76.0 2.84 76.2 2.90 br t

Xyl-4 69.0 3.07 69.7 3.02

Xyl-5 65.7 3.07, 2.33 65.8 3.02, 2:39

1. Method for producing a plant extract, quantified on pinitol, wherein a plant Desmodium adscendens is selected from the Desmodium family, wherein a fraction is extracted from Desmodium plant parts, wherein a plant extract is derived from said fraction thereof, characterized in that a characterised extract is derived from Desmodium adscendens, from which a preparation of said plant extract is quantified on pinitol.

2. Method according to claim 1, characterized in that a validated analytical method is performed for said quantification of Desmodium adscendens on pinitol, in particular D-pinitol, wherein a specific, quantified D. adscendens preparation is produced as “totum”.

3. Method according to the preceding claim, characterized in that a biologically controlled isolation is carried out in said analytical method which is carried out with generating a composition quantified for D-pinitol, wherein the composition product is repeatedly enriched and numerous fractions are isolated, and wherein a decoction is obtained that contains a significant amount of D-pinitol.

4. Method according to any one of the preceding claims, characterized in that the constituents of said composition are standardised with capture of the reproducibility of the production process.

5. Method according to any one of the claims 2 to 4, characterized in that the main components of the constituents in Desmodium adscendens, and the total amount of D-pinitol are determined by means of a suitable analytical method therefor, in particular wherein the extraction process, the products obtained after extraction and the conditions are evaluated and optimised.

6. Method according to any one of the preceding claims, characterized in that said plant extract is derived from a fraction consisting of above-ground parts of Desmodium adscendens, either including leaves, branches, stems and/or flowers or fruits thereof.

7. Method according to any one of the preceding claims, characterized in that said plant extract is derived from a fraction consisting of above-ground parts of Desmodium adscendens that are seasonal, either including flowers or fruits, optionally seeds thereof.

8. Method according to any one of the preceding claims, characterized in that said plant extract is derived from a fraction consisting of under-ground parts of Desmodium adscendens thereby including roots thereof.

9. Method according to the preceding claim, characterized in that said plant parts are first dried.

10. Method according to any one of the preceding claims, characterized in that said plant parts are then ground or fragmented.

11. Method according to the preceding claim, characterized in that said plant parts are pulverized into powder form.

12. Method according to the preceding claim, characterized in that said powder is converted as such into a pharmaceutical form.

13. Method according to one of the preceding claims, characterized in that an extract of said plant parts of Desmodium adscendens is prepared by extraction with lower alkyl alcohols.

14. Method according to the preceding claim, characterized in that an extract of said plant parts of Desmodium adscendens is prepared by extraction with lower alkyl alcohols including methanol, ethanol and/or isopropanol, or combinations thereof.

15. Method according to one of both preceding claims, characterized in that an extract of said plant parts of Desmodium adscendens is prepared by extraction with water.

16. Method according to one of both claim 13 or 14, characterized in that an extract of said plant parts of Desmodium adscendens is prepared by extraction with less polar or non-polar solvents such as ethyl acetate or n-hexane, or combinations thereof.

17. Method according to any one of the claim 13 or 14, characterized in that an extract of said plant parts of Desmodium adscendens is prepared by extraction with using solvents under supercritical conditions, such as carbon dioxide.

18. Method according to any one of the preceding claims, characterized in that an aqueous decoction of said plant parts of Desmodium adscendens is then prepared warm to a decoct by boiling a certain quantity of dried, optionally powdered, leaves in a certain quantity of water, in particular distilled water, for a certain period of time.

19. Method according to the preceding claim, characterized in that said aqueous decoction of said plant parts of Desmodium adscendens is then cooled, after which the portions obtained are combined and filtered, after which the filtrate obtained is concentrated, in particular under vacuum, and then lyophilized to form a lyophilizate.

20. Method according to claim 18, characterized in that said aqueous decoction of said plant parts of Desmodium adscendens is then cooled, after which the resulting portions are pooled and filtered, after which the filtrate is concentrated, in particular under vacuum, and then spray-dried.

21. Method according to any one of the claims 1 to 17, characterized in that an aqueous of product as macerate of said plant parts of Desmodium adscendens is then cold-processed to an extract, in particular by incorporating a certain amount of dried, optionally pulverized plant parts of to be included in a certain amount of water, in particular distilled water, for a certain period of time.

22. Method according to any one of claims 9 to 21, characterized in that starting from 1 kg of dried plant parts, there is obtained from 60 to 70 g, in particular about 65 g of extract, respectively according to a ratio of 10-20:1, more particularly 14-16:1.

23. Method according to any one of claims 7 to 22, characterized in that a certain quantity of said decoction is then subjected to column chromatography, with methanol elution, wherein certain fractions are collected and analysed by thin-layer chromatography, and MeOH/H₂O:5:1 as a mobile phase, and wherein their chromatographic pattern is determined.

24. Method according to the preceding claim, characterized in that about 20 g of said extract is subjected to column chromatography on Sephadex LH-20 with methanol elution, whereby about 100 ml is collected and analyzed by thin layer chromatography, silica gel with a layer thickness of approximately 0.25 cm, and MeOH/H2O: 5:1 as mobile phase, and wherein their chromatographic pattern is determined on the basis of these parameters.

25. Method according to one of both preceding claims, characterized in that said fractions are combined into a number of sub-fractions, in particular 11, according to their chromatographic pattern.

26. Method according to the preceding claim, characterized in that said sub-fractions 5-11, in particular 200 mg, which have a spot with a green color, are combined, after spraying with anisaldehyde 1% H2SO4 in MeOH and heating to about 120° C. for 10 min, wherein said fraction is subjected to further column chromatography eluted with MeOH, wherein certain fractions, in particular of 100 ml, are once again collected and analysed.

27. Method according to the preceding claim, characterized in that said sub-fractions 4-5, in particular 120 mg, from this column are combined, wherein the latter are subjected to a further column chromatography under the same conditions, after which a pure product is obtained, in particular white.

28. Method according to any one of the claims 3 to 27, characterized in that said extract is separated and the isolated product is identified as the methylated cyclitol 3-O-methyl-chiro-inositol, i.e. D-pinitol.

29. Method according to the preceding claim, characterized in that sugar alcohols are selected as an internal standard, in particular xylitol, notably in gas chromatography.

30. Method according to the preceding claim, characterized in that also other sugar alcohols are selected such as sorbitol, mannitol, optionally dulcitol and inositol, instead of the abovementioned xylitol, with the latter two, in a lesser extent.

31. Method according to any one of the preceding claims, characterized in that L-pinitol is generated from said extract from which it is separated.

32. Method according to any one of the preceding claims, characterized in that one starts from a plant extract of Desmodium adscendens of tropical origin.

33. Method according to any one of claims 1 to 31, characterized in that one starts from a plant extract of Desmodium adscendens of cultured origin.

34. Method according to any one of the preceding claims, characterized by an extension of the characterization of the Desmodium adscendens preparation with a flavonoid profile.

35. Method according to the preceding claim, characterized in that vitexin is identified wherein the total amount of flavonoids, defined as the sum of the signals obtained, expressed as vitexin, amounts to approximately 1.05%, in a typical lyophilizate of the decoct of D. adscendens, as flavonoid rate and profile in Desmodium adscendens lyophilisate between 0.1 and 5%, in particular wherein said rate is of the order of 1%.

36. Method according to any one of the claims 1 to 35, characterized in that a standardized extract is derived from the plant Desmodium adscendens, quantified for D-pinitol in its action against hepatitis, in particular in preventive action, with the separation of D-pinitol from Desmodium adscendens, wherein D-pinitol forms an active ingredient.

37. Method according to any one of the claims 1 to 35, characterized in that a standardized extract is derived from the plant Desmodium adscendens, quantified on D-pinitol in its curative effect against hepatitis, with isolation of D-pinitol from Desmodium adscendens, wherein D-pinitol forms an active constituent.

38. Method according to one of both preceding claims, characterized in that a standardized extract is derived from the plant Desmodium adscendens, quantified for D-pinitol in its activity against hepatitis, with regard to liver damage of chemical, physical, infectious or immunological origin.

39. Plant extract obtained according to a method as defined in one of the preceding claims for use in a method for protecting the liver in a mammal, for the prevention of a liver disease in a mammal.

40. Plant extract obtained according to a method as defined in any one of claims 1 to 35 for treatment of a liver disease in a mammal.

41. Plant extract according to any one of both preceding claims, characterized in that it is to be derived from plant Desmodium adscendens, quantified on the molecule D-pinitol active against hepatitis, wherein D-pinitol is isolated from Desmodium adscendens D-pinitol of which on an active ingredient, standardized forms.

42. Plant extract according to any one of the claims 39 to 41, characterized in that said plant extract contains between 0.1 and 10%, preferably from 4 to 5% pinitol, in particular D-pinitol.

43. Plant extract according to any one of the claims 39 to 42, characterized in that said D-pinitol containing plant extract is given to a mammal in the form of a composition containing the latter, wherein said composition is selected from the group consisting of a pharmaceutical composition, a food composition and/or a beverage composition.

44. Extract from a plant Desmodium adscendens according to any one of claims 39 to 43, for use in a method of prevention of a liver disease in a mammal, which comprises the step consisting in the administration of an effective amount of said extract of Desmodium adscendens thereto.

45. Extract from Desmodium adscendens according to any one of the claims 39 to 44, for use in a method of treatment of a liver disease in a mammal, which comprises the step consisting in the administration of an effective amount of the abovementioned extract of Desmodium adscendens thereto.

46. Use of a vegetable extract as defined in any one of the claims 39 to 45, characterized in that this is employed as an anti-oxidizing agent intended for specific target groups, in particular where the target referred formed by man, more particularly alcoholics and/or by patients with a metabolic syndrome, yet more particularly by diabetes patients with type 2 diabetes.
